(19) United States
(12) Patent Application Publication
(10) Pub. No.: US 2009/0062871 A1
(43) Pub. Date: Mar. 5, 2009

(54) BALLOON CANNULA SYSTEM FOR ACCESSING AND VISUALIZING SPINE AND RELATED METHODS

(76) Inventors: Singfatt Chin, Pleasanton, CA (US); John T. To, Newark, CA (US); Lex P. Jansen, Pleasanton, CA (US); Myra I.L. Fabro, San Jose, CA (US)

Correspondence Address: MORRISON & FOERSTER LLP, 755 PAGE MILL RD, PALO ALTO, CA, 94304-1018, US

(21) Appl. No.: 12/199,701

(22) Filed: Aug. 27, 2008

Related U.S. Application Data
(60) Provisional application No. 60/968,086, filed on Aug. 27, 2007.
(60) Provisional application No. 61/045,919, filed on Apr. 17, 2008.

(51) Int. Cl. A61F 5/00; A61B 1/00; A61M 31/00; A61M 29/00

(52) U.S. Cl. 606/86 R; 600/106; 604/509; 606/192

(57) ABSTRACT

Balloon cannula systems may be used for accessing and visualizing the spine and related methods of treatment, including a forward-looking balloon system for creating a working space and the balloon system having atraumatic dissection capability to allow visualization in spine. The devices and methods described may be used, for example, to perform annulus repair, herniated disc excision, and denervation of neurological tissue; to dispense pharmacological agents and/or cell or tissue therapy agents; to diagnose disc degeneration and bony degeneration, spinal stenosis, and nucleus decompression, and to perform disc augmentation.

### CROSS-REFERENCE TO RELATED APPLICATIONS

The present application claims priority under 35 U.S.C. §119(e) to U.S. Provisional Application Ser. No. 60/968,086, filed Aug. 27, 2007, and U.S. Provisional Application Ser. No. 61/045,919, filed Apr. 17, 2008, all of which are hereby incorporated by reference in their entirety.

### BACKGROUND OF THE INVENTION

Injured intervertebral discs are generally treated with bed rest, physical therapy, modified activities, and pain medications for substantial treatment durations. There are also a number of treatments that attempt to repair injured intervertebral discs and to avoid surgical removal of injured discs. For example, disc decompression is a procedure used to remove or shrink the nucleus, thereby decompressing and decreasing the pressure on the annulus and nerves. Less invasive procedures, such as microlumbar discectomy and automated percutaneous lumbar discectomy, remove the nucleus pulposus of a vertebral disc by aspiration through a needle laterally inserted into the annulus. Another procedure involves implanting a disc augmentation device in order to treat, delay, or prevent disc degeneration. Augmentation refers to both (1) annulus augmentation, which includes repair of a herniated disc, support of a damaged annulus, and closure of an annular tear, and (2) nucleus augmentation, which includes adding material to the nucleus. Many conventional treatment devices and techniques, including open surgical approaches, involve muscle dissection or percutaneous procedures to pierce a portion of the disc under fluoroscopic guidance, but without direct visualization. Several treatments also attempt to reduce discogenic pain by injecting medicaments or by lysing adhesions in the suspected injury area. However, these devices also provide little in the form of tactile sensation for the surgeon or allow the surgeon to atraumatically manipulate surrounding tissue. In general, these conventional systems rely on external visualization for the approach to the disc and thus lack any sort of real time, on-board visualization capabilities.

Furthermore, accurately diagnosing back pain is often more challenging than many people expect and often involves a combination of a thorough patient history and physical examination, as well as a number of diagnostic tests. A major problem is the complexity of the various components of the spine, as well as the broad range of physical symptoms experienced by individual patients. In addition, the epidural space contains various elements such as fat, connective tissue, lymphatics, arteries, veins, blood, and spinal nerve roots. These anatomical elements make it difficult to treat or diagnose conditions within the epidural area because they tend to collapse around any instrument or device inserted therein. This may reduce visibility in the epidural space, and may cause inadvertent damage to nerve roots during device insertion. Also, the insertion of a visualization device may result in blocked or reduced viewing capabilities. As such, many anatomical elements within the epidural space may limit the insertion, movement, and viewing capabilities of any access, visualization, diagnostic, or therapeutic device inserted into the epidural space.

### BRIEF SUMMARY OF THE INVENTION

Some embodiments relate to balloon cannula systems for accessing and visualizing the spine and related methods of treatment, including a forward-looking balloon system for creating a working space and the balloon system having atraumatic dissection capability to allow visualization in spine. The devices and methods described herein may be used, for example, to perform annulus repair, herniated disc excision, and denervation of neurological tissue. The devices and methods may also be used to dispense pharmacological agents and/or cell or tissue therapy agents, to diagnose disc degeneration and bony degeneration, spinal stenosis, and nucleus decompression, and to perform disc augmentation.

In one embodiment, there is provided a method of accessing a portion of the spine including percutaneously approaching a portion of the spine with an instrument having direct visualization capability, providing an access to a portion of the spine using the instrument, and delivering a device into the access provided using the instrument. In a further embodiment, the method includes delivering an expandable structure adjacent a portion of the spine to be accessed and expanding the expandable structure. In another embodiment, the expandable structure is a balloon or an expandable atraumatic element and may contain a material or marker to enhance visualization of the structure using an imaging modality outside of the body. In another embodiment, the device to be delivered is a monitor, a therapy delivery device, a stimulation device or a pharmacological therapy device or, alternatively, the device comprises an electrode, and wherein providing an access to a portion of the spine comprises providing an access to the spinal epidural space. In another embodiment, the method includes implanting the device using the direct visualization capability of the instrument. In still another embodiment, expanding the expandable structure comprises atraumatically deforming a portion of the spinal dura mater to create a sufficient working space. In still other embodiments, a method includes providing an access to a portion of the spine, such as, providing an access to the spinal epidural space, the annulus, the layers of annulus, the disc nucleus, the facet joints, the foramen, or the muscle. In still another embodiment, the method also includes receiving visualization information from an imaging modality outside of the body such as, for example, from fluoroscopy, magnetic resonance imaging, and/or computer tomography. In still other embodiments, the method includes using the direct visualization capability of the instrument to maneuver the instrument between a spinal nerve root, the spinal dura and nerve tissue and other soft tissue, to atraumatically manipulate the spinal nerve root or other soft tissue and/or advancing the instrument while using a portion of the instrument to atraumatically manipulate the spinal nerve root or other soft tissue. In yet another embodiment, the method includes using the subject devices to deliver disc augmentation devices or nucleus augmentation devices or disc excision devices. In another embodiment, the balloon cannula device may be used for diagnostic purposes.

In one embodiment, a balloon cannula system (access system) is fitted with an extrusion (e.g. deflated balloon material) that is terminally bonded. Following positioning of the balloon cannula system at the targeted site to be treated, the balloon may be inflated and may be used as an atraumatic tool for dissection and/or an atraumatic tool to create working space, thereby enhancing visualization of the surrounding structures. In one embodiment, the balloon is a forward-looking structure so that the distal tip of the balloon may push obstructive tissue away from the scope, and the distal tip of the balloon may provide a depth of view between the scope and the targeted sites to be treated.

One embodiment is directed to a balloon cannula device comprising a multi-lumen elongated shaft, a balloon attached at its distal end of the shaft, wherein the proximal end of the balloon and distal end of the balloon are attached to the outer surface of the same elongated shaft, and wherein the balloon is constructed such that following inflation of the balloon, said balloon is forward-looking to create a working space distally to the viewing scope to enhance direct visualization. In another embodiment, the balloon of the balloon cannula system includes at least one portion that is elastically expandable. The expandable balloon may be inflated with air, sterile saline, contrasting agent, or other agents that may be delivered via a syringe or a pump. In some embodiments, the balloon is able to simultaneously undergo radial expansion and keep the forward-looking feature of the balloon cannula system. In one or more of the embodiments described herein, the distance between the points of attachment of the balloon to the same outer shaft of the elongated shaft is between about 1 mm and about 15 mm. In another embodiment, one end of the balloon is attached to a distal end of the balloon catheter in a flipped manner (e.g. everted or inverted), such that the internal surface of the balloon is in contact with the elongated shaft distally, and the outer surface of the balloon is in contact with the same elongated shaft proximally. In yet another embodiment, the balloon includes at least one elastically deformable portion. In yet another embodiment, the deformable portion is constructed of polyurethane.

Some embodiments also provide a balloon catheter having a proximal portion and a distal portion and one or more lumens, wherein said proximal portion contains 3 separate lumens, one of said lumens being suitable for allowing the passage of an endoscope, one of said lumens being suitable for inflation of a balloon, and the other lumen being suitable for allowing passage of therapeutic instruments or injection of medications. The distal portion of the balloon catheter may be a dual-lumen conduit, comprising an inflation lumen in communication with said lumen in the proximal portion and suitable for inflation of the balloon, and a common lumen in communication with the lumens of the proximal portion suitable for passage of the endoscope and the lumen suitable for allowing passage of therapeutic instruments or injection of medications. The balloon may comprise a balloon material attached at its distal end to the outer surface of said distal portion of the balloon catheter, and/or wherein at least part of said distal portion of the balloon catheter is constructed such that following inflation of the balloon, said balloon is forward-looking to create a working space distally to the viewing scope to enhance direct visualization. In another embodiment, the distal portion of the balloon catheter includes at least one portion that is elastically deformable when inflated. In yet another embodiment, at least one elastically deformable portion comprises polyurethane.

In another embodiment, an apparatus and method for treating spinal disorders in a patient in need of such treatment, includes introducing a balloon cannula device having direct visualization capability into the patient, steering the balloon cannula device to a position adjacent an outer surface of the spinal targeted site using visualization information provided by an endoscope or other visualization device in combination with the balloon cannula device, dissecting and/or displacing tissue with the balloon on the balloon cannula device to create a working space, and using the balloon cannula device to deliver a disc augmentation device for treating disc degeneration.

In one embodiment, a method for treating intervertebral disc degeneration in a spine includes introducing a balloon cannula device that permits direct visualization capability into a portion of the spine, inflating the balloon cannula to create a forward-looking capability to enhance visualization and displacement of tissues, and introducing a therapy device into the balloon cannula device to treat disc degeneration.

In another embodiment, a method for treating intervertebral disc degeneration in a spine of a body includes making an incision into a skin of the body, introducing a balloon cannula device that permits direct visualization into a portion of the spine, inflating the balloon cannula to create a forward-looking capability that enhances visualization and displacement of tissues, introducing a therapy device into balloon cannula device to treat disc degeneration, and treating the disc degeneration.

In another embodiment, a method for treating intervertebral disc degeneration includes introducing a balloon cannula device that permits direct visualization capability into a portion of the spine, steering the balloon cannula device to a position adjacent to an outer surface of the disc or nervous tissues using visualization information provided by a visualization system, displacing the nervous tissues or other tissues with a portion of the balloon cannula device to create a working area, using the balloon cannula device to deliver a therapy device for treating intervertebral disc degeneration, and treating the disc degeneration. The visualization system may be used in conjunction with the balloon cannula device or may be integrated with the balloon cannula device. In some embodiments, the therapy device is a nucleus decompression device configured to remove a portion of the nucleus, the annulus, or one or more fragmented segments of the vertebral disc. In some embodiments, a therapy device shrinks a portion of the nucleus or the annulus. Treating the disc degeneration may also comprise repairing a herniated disc, supporting a damaged annulus, adding material to the nucleus or annulus, and/or sealing an annulus. In one embodiment, displacing the tissues comprises expanding an expandable structure of the balloon cannula device.

In another embodiment, a system for intervertebral disc augmentation includes a balloon cannula device configured to deliver a disc augmentation device to an intervertebral disc. In one embodiment, the balloon cannula device includes an elongate body, an expandable structure, a direct visualization device, and at least one working channel. The expandable structure may be a mesh, a balloon, an atraumatic element, or a combination thereof. In one or more of the embodiments, the expandable structure may be configured to deform a portion of the spinal dura mater or tissues in the spinal area, and to create a working area. In one or more of the embodiments, the expandable structure comprises a forward-looking balloon. A direct visualization device inserted into the balloon cannula device or integral with the balloon cannula device may provide visualization information from an image generated by a sensor located on the direct visualization device. In some embodiments, the augmentation device comprises at least one mesh, cage, barrier, patch, scaffold, sealing means, hydrogel, silicone, growth factor, or combination thereof. In some embodiments, the augmentation device is an ablation device, a grasper or forceps, or a temperature-controlled energy element, for example. The energy element may be a thermal energy device that delivers resistive heat, radiofrequency, coherent and incoherent light, microwave, ultrasound or liquid thermal jet energies to the nucleus.

In another embodiment, a method of diagnosing disc degeneration in a patient includes introducing a balloon cannula device permitting direct visualization capability into a portion of the spine, steering the balloon cannula device using visualization information provided by the balloon cannula device, displacing the nervous tissues or other tissues with a portion of the balloon cannula device to create a working area, and assessing the condition of targeted site. The balloon cannula device may comprise a material or marker to enhance visualization of the structure using an imaging modality outside of the body. The method may include receiving visualization information from an imaging modality outside of the body. The imaging modality may comprise fluoroscopy and/or magnetic resonance imaging. The visualization information may be provided from an image generated by a sensor located on the visualization device. The balloon cannula device may also include a sensor for collecting diagnostic data.

In another embodiment, a kit for augmenting the intervertebral disc may include at least one disc augmentation device, a balloon cannula device having a forward-looking balloon at its distal tip, an endoscopic mechanism having direct visualization capabilities, and instructions for locating the at least one disc augmentation device using the balloon cannula device. The kit for decompressing the nucleus of an intervertebral disc may also include at least one nucleus decompression device, a balloon cannula device having a forward-looking balloon at its distal tip that permits direct visualization using an endoscope or other visualization device, and instructions for decompressing the nucleus of an intervertebral disc using the balloon cannula device.

In another embodiment, a method for treating intervertebral disc degeneration includes introducing a balloon cannula device permitting direct visualization into a portion of the spine using a visualization mechanism, displacing the spinal column matter with a portion of the balloon cannula device to create a working area, and using the balloon cannula device to deliver a stimulation electrode device for treating intervertebral disc degeneration. In one or more of the embodiments, the balloon cannula device may be steered to a position within the spinal column using the direct visualization of a visualization mechanism positioned within the balloon cannula device. The method may also include introducing a balloon cannula device permitting direct visualization into a portion of the spine using a visualization mechanism, steering the balloon cannula device using visualization information provided by the visualization mechanism, displacing the tissues in spinal area with a portion of the balloon cannula device to create a working area, and using the balloon cannula device to deliver a stimulation electrode device for treating intervertebral disc degeneration. The visualization mechanism, such as an endoscope, may be placed into the balloon cannula device or may be integrally formed with the balloon cannula device.

In another embodiment, a balloon cannula device for assessing a target site within the body may include a multi-lumen elongated shaft and a balloon attached at a distal end of the shaft, wherein the proximal end and distal end of the balloon are attached to the outer surface of the elongated shaft and wherein the balloon is constructed such that following inflation of the balloon, the balloon is forward-looking and create a working space distally to the viewing scope to enhance direct visualization.

In another embodiment, a balloon cannula device for visualizing a target site within body may include a proximal portion and a distal portion, at least three lumens positioned within the proximal portion, wherein at least one lumen is suitable for allowing the passage of endoscope, at least one lumen is suitable for inflation of a balloon, and at least one lumen is suitable for allowing passage of therapeutic instruments or injection of medications. In some embodiments, at least two lumens may be positioned within the distal end, and at least one of the lumens permits visualization of therapeutic instruments or injected medications. A balloon may be attached to an outer surface of the distal portion of the balloon cannula device, and at least part of the distal portion of the balloon cannula device may be constructed such that following inflation of the balloon, the balloon is forward-looking to create a working space distally to enhance direct visualization. In one or more of the embodiments described herein, the balloon is constructed of polyurethane, but in other embodiments may be constructed of a polymer material other than polyurethane.

In another embodiment, a balloon cannula device for visualizing a target site within body may include an elongated shaft having a proximal portion and a distal portion, wherein the proximal portion contains four separate lumens, one of said lumens being suitable for allowing the passage of the endoscope and/or irrigation therethrough, one of said lumens being suitable for allowing the passage for therapeutic instruments and/or aspiration, one of the said lumens being suitable for inflation of the balloon, and one of said lumens for additional aspiration or irrigation. The distal portion of the balloon cannula device may contain three lumens, with one of said lumens being the continuation of the lumen for the endoscope and/or irrigation, one of said lumens being the continuation of the lumen for therapeutic instruments and/or aspiration, and one of said lumens being the continuation of lumen for additional aspiration or irrigation. The balloon may be attached at its distal end of the balloon cannula device, in such a way that one end of the balloon is attached at its distal end in an flipped manner, with the internal surface of said balloon in contact with the catheter shaft distally, and the outer surface of said balloon in contact with the same catheter shaft proximally. The distal portion of the device may be constructed such that following inflation of the balloon, the balloon is able to simultaneously undergo radial expansion and keep the forward-looking aspect. The use of any one lumen need not be limited to a particular instrument or procedure, and may be used differently from the exemplary embodiments disclosed herein. In some embodiments, two or more lumens may be used for the same purpose during a procedure.

In one embodiment, a minimally invasive spinal endoscopy system is provided, comprising a tubular shaft with a slotted flexion zone, at least two slidable control wires, a proximal end, a distal end, at least two irrigation channels, an inflation channel, at least one non-circular instrument channel, and a visualization channel. In some examples, the tubular shaft may have an average diameter of less than about 3.5 mm. The system may further comprise a movable actuator attached to at least two slidable control wires, a housing enclosing the proximal end of the tubular shaft and at least a portion of the movable actuator, and an inflatable balloon. The balloon may comprise an extruded tubular polymeric material comprising a folded section and post-extrusion reoriented polymer chains, a proximal attachment to the tubular shaft and a distal attachment to the tubular shaft, wherein the spacing between the proximal attachment and the distal attachment has a fixed distance, a proximal end that is proximal to the distal end of the shaft, a distal end that is distal to the distal end of the tubular shaft, an open-ended common balloon lumen between the distal end of the shaft and the distal end of the inflatable balloon, wherein the open-ended common balloon lumen has a length of at least 1 mm and is in communication with at least two irrigation channels and at least two non-circular instrument channels, and a balloon cavity in communication with the inflation channel of the tubular shaft, wherein the inflatable balloon has a substantially cylindrical uninflated configuration and a substantially toroidal inflated configuration having a diameter that is about three to about six times greater than the longitudinal length of the open-ended common balloon lumen when the inflatable balloon is inflated to at least about 60 psi, wherein the average diameter of the open-ended common balloon lumen decreases by less than about 15% from the uninflated configuration to the inflated configuration at a pressure of at least about 60 psi. The minimally invasive spinal endoscopy system may further comprise an endoscope with a shaft having an average diameter of less than about 1 mm and configured for insertion into the visualization channel. In some examples, the visualization channel may have a smaller cross-sectional area than at least one instrument channel. The minimally invasive spinal endoscopy system may also further comprise a guidewire, a dilator, an introducer sheath, a tissue debrider, a grasper, a coagulation probe, and an infusion cannula configured for insertion into at least one instrument channel.

In another embodiment, a minimally invasive device for use in a body is provided, comprising a tubular body comprising a proximal end, a distal end, a first lumen therebetween, and an inflation lumen, and an inflatable member with an inflation chamber in communication with the inflation lumen of the inflatable member, a proximal end, a distal end, a balloon lumen therebetween that is in communication with the first lumen of the tubular body. The proximal end of the inflatable member may be proximal to the distal end of the tubular body and the distal end of the inflatable member may be distal to the distal end of the tubular body. The inflatable member may also have a base unexpanded configuration and an expanded configuration. The inflatable member may be configured to return toward the unexpanded configuration when deflated from the expanded configuration. In some examples, the inflatable member may comprise a biaxially oriented material, including an extruded polymeric material with post-extrusion reoriented polymer chains. The system may be configured such that the average cross-sectional area of the second lumen changes less than 10 percent between the unexpanded configuration and the expanded configuration. The device may also further comprise a second lumen between the proximal end and the distal end of the tubular body, wherein the second lumen is in communication with the balloon lumen of the inflatable member. The first lumen may also have a non-circular shape. In certain steerable embodiments, the tubular body further comprises at least one deflection wire and a flexion plane. The first lumen of the tubular body comprises a first central axis, the second lumen of the tubular body may comprise a second central axis, and the first central axis and the second central axis are located generally along a plane perpendicular to the flexion plane of the tubular body. The inflatable member may also comprise a toroidal shape. In some further embodiments, the distance between the proximal end of the inflatable member and the distal end of the tubular body may be about three times to about seven times greater than a distance between the distal end of the inflatable member and the distal end of the tubular body, but in other embodiments, may be about four times to about six times greater than a distance between the distal end of the inflatable member and the distal end of the tubular body. The device may also comprise a cannula with a distally extending inflatable member with a through lumen, wherein the inflatable member is sealed to the cannula to withstand an inflation pressure of at least about 40 psi, or even at least about 60 psi.

In one embodiment, a kit for performing a medical procedure may be provided, comprising a cannula comprising a cannula lumen and a distally extending inflatable member with a through lumen, and a rotatable tissue removal device configured for insertion through the cannula and into the through lumen of the distally extending inflatable member. The kit may also further comprise an endoscope configured for insertion into the cannula.

In another embodiment, a method for minimally invasively accessing a body site is provided, comprising providing a tubular body with an inflatable member located at a distal end of the tubular body and protruding distally from the distal end of the tubular body, wherein the inflatable member has a common lumen, an unexpanded configuration and an expanded configuration, inserting the tubular body toward a non-vascular target site in a body, inflating the inflatable member to the expanded configuration while in the body, and visualizing the non-vascular target site from the tubular body and through the common lumen of the distally protruding inflatable member. The method may also optionally comprise inserting an endoscopic device into the tubular body. The endoscopic device may or may not be inserted into the through lumen of the inflatable member. The method may also include advancing the distal end of the tubular body toward a neural structure in contact with a non-neural structure, displacing the neural structure from the non-neural structure using the inflatable member, and/or orienting the common lumen of the inflatable member with the non-vascular target site.

In another embodiment, a method of manufacturing a medical component is provided, comprising providing a first tubular body comprising a proximal end and a distal end, providing a second tubular body comprising a proximal end, a distal end and an intermediate section therebetween, attaching the proximal end of the second tubular body at a first attachment site proximal to the distal end of the first tubular body while positioning the distal end of the second tubular body distal to the distal end of the first tubular body, and attaching the distal end of the second tubular body to the first tubular body so that at least a portion of the intermediate section of the second tubular body is distal to the distal end of the first tubular body. In some embodiments, the second tubular body may be cylindrical and/or may be an extruded tubular body. In some examples, the method may further comprise pressurizing the second tubular body while the second tubular body is heated (e.g. to a temperature of at least 110 degrees F.), which may be followed by cooling the second tubular body while the second tubular body is pressurized. In other examples, the second tubular body may be inserted into a third tubular body before pressurizing the second tubular body. In some instances, the proximal end and the distal end of the second tubular body may be sealed to the first tubular body to withstand an inflation pressure of at least about 40 psi without significant separation of the second tubular body from the first tubular body. The attachment of the distal end of the second tubular body may occur before or after attaching the proximal end of the second tubular body.

Another embodiment comprises a method for treating intervertebral disc degeneration in a spine, which may involve introducing a balloon cannula device having direct visualization capability into a portion of a spine, inflating the balloon cannula to create a forward looking capability to enhance visualization and displacement of tissues, and introducing a therapy device into the balloon cannula device to treat disc degeneration. The therapy device may have a variety of configurations, including to configuration that provide structural support to a disc annulus of the spine, those that can seal a torn annulus, and/or those that add or remove additional material to the nucleus.

In some embodiment, a method for treating intervertebral disc degeneration in a spine of a body may comprise making an incision into a skin of the body, introducing a balloon cannula device having direct visualization component into a portion of the spine, inflating the balloon cannula to create a forward looking capability to enhance visualization and displacement of tissues, introducing therapy device into balloon cannula device to treat disc degeneration, and treating the disc degeneration.

In another embodiment, a method for treating intervertebral disc degeneration may comprise introducing a balloon cannula device having direct visualization capability into a portion of the spine, steering the balloon cannula device to a position adjacent an outer surface of the disc or nervous tissues using visualization information provided by the balloon cannula device, displacing the nervous tissues or other tissues with a portion of the balloon cannula device to create a working area, using the balloon cannula device to deliver a therapy device for treating intervertebral disc degeneration, and treating the disc degeneration. The therapy device may be a nucleus decompression device to remove a portion of the nucleus, annulus, or fragmented segments, or a therapy device shrinks a portion of the nucleus or annulus, for example. More than one therapy device may be provided or used with the balloon cannula device. Treatment of the disc degeneration may comprise repairing a herniated disc, supporting a damaged annulus, sealing an annulus, adding material or removing material with respect to the nucleus or annulus, and/or expanding an expandable structure of the balloon cannula device.

### BRIEF DESCRIPTION OF THE DRAWINGS

The invention is best understood from the following detailed description when read in conjunction with the accompanying drawings. It is emphasized that, according to common practice, the various features of the drawings may or may not be to-scale. On the contrary, the dimensions of the various features may be arbitrarily expanded or reduced for clarity. Included in the drawings are the following figures:

FIG. 1 is a perspective view of a balloon cannula device, wherein the balloon inflated.

FIG. 2 is a perspective view of a distal portion of the balloon cannula device, wherein the balloon is inflated.

FIG. 3 is a perspective view of an alternative view of the distal portion of the balloon cannula device.

FIG. 4 is a cross-sectional view of the balloon in a stowed condition (uninflated).

FIG. 5 is a cross-sectional view of the balloon in a deployed condition (inflated).

FIG. 6 is a cross-sectional view of a balloon formed in the deployed condition.

FIG. 7 is a cross-sectional view of the balloon (inflated) attached to the shaft of the balloon cannula device. One end of the balloon is attached at its distal end in a flipped manner, such that the internal surface of the balloon is in contact with the catheter shaft distally, and the outer surface of the balloon is in contact with the same catheter shaft proximally.

FIG. 8 is a cross-sectional view of a multi-lumen extrusion for a disc augmentation application.

FIG. 9 is a cross-sectional view of a multi-lumen extrusion for a thermal denervation application.

FIG. 10 is a cross-sectional view of a multi-lumen extrusion for a selective nerve block application.

FIGS. 11A and 11B are cross-sectional views of one embodiment of a balloon cannula tip with a non-expandable distal segment, before and after inflation, respectively.

FIGS. 12A and 12B are cross-sectional views of another embodiment of a balloon cannula tip with a non-expandable distal segment, before and after inflation, respectively.

FIG. 13 is a cross-sectional view of a cannula tip with a non-expandable atraumatic tip.

FIG. 14 is a schematic cut-away view of the housing of one embodiment of a balloon cannula device.

FIGS. 15A to 15C are detailed views of various embodiments of a cannula device with a steering mechanism.

FIG. 16 depicts one embodiment of a flex region of a cannula device.

FIG. 17A depicts another embodiment of a flex region of a cannula device;

FIG. 17B is a detailed schematic view of a flex region during flexion.

FIG. 18 depicts another embodiment of a flex region of a cannula device.

FIGS. 19A and 19B are schematic cross-sectional views of a balloon cannula device with an inserted endoscope in a neutral and a flexed position, respectively.

FIG. 20 is a schematic representation of one embodiment of a tubular shaft of a cannula device in a neutral position and in various flexed positions within a bending plane (depicted with dashed lines).

FIG. 21 is a schematic representation of one embodiment of a cannula device with two channels centered along a plane perpendicular to a bending plane of the cannula device.

FIGS. 22A and 22B are cut-away and side elevational views of one embodiment of a balloon cannula device with an endoscopic coupling port, respectively

FIG. 23 is a cut-away view of the balloon cannula device of FIG. 14 with tubes connected to the tubular shaft.

FIG. 24 is a side elevational view of the balloon cannula device of FIG. 23.

FIG. 25 is a side elevational view of another embodiment of a balloon cannula.

FIG. 26 is a schematic side cut-away view of one approach to the vertebrae.

FIG. 27 is a schematic superior cut-away view of one approach to the vertebrae.

FIG. 28 is an isometric view of another embodiment of a balloon cannula device with a conical balloon.

FIG. 29 is a cross-sectional view of another embodiment of a balloon cannula device.

FIG. 30 is a cross-sectional view of an embodiment of a balloon cannula device comprising multiple balloons.

### DETAILED DESCRIPTION OF THE INVENTION

Conventional systems often rely on external visualization such as fluoroscopy and CT scanning for the approach to the disc, and thus lack any sort of real time, on-board visualization capabilities. Also, existing devices provide little in the form of tactile sensation for the surgeon and do not allow the surgeon to atraumatically manipulate surrounding tissue.

There is a need, therefore, for minimally invasive techniques and systems that provide the capability to diagnose or repair the spine using direct visualization while minimizing damage to surrounding anatomical structures and tissues. There is also a need for a method and device that allows a physician to effectively enter the epidural space of a patient, clear an area within the space to enhance visualization and use the visualization capability to diagnose and treat the disc injury.

The embodiments disclosed herein will be more clearly understood and appreciated with respect to the following Detailed Description, when considered in conjunction with the accompanying Drawings.

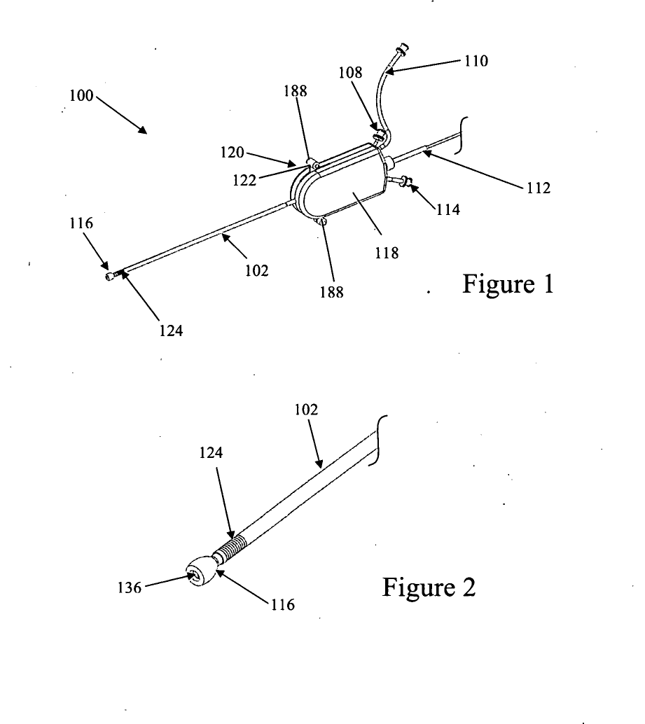

FIGS. 1 to 3 are different views of one embodiment of a balloon cannula device 100, which may comprise a tubular shaft 102 with a proximal end 104 and a distal end 106. The proximal end of the shaft 102 may be associated with one or more ports 108, 110, 112, and 114, and the distal end 106 may be coupled to a distal expandable member, including but not limited to an inflatable balloon 116. The balloon 116 may be used to create working space, dissect or deform or manipulate surrounding tissue, structure, or anatomical features, for example. The balloon 116 may also be used to provide a forward-looking or forward-separating feature for the endoscope to effectively visualize targeted sites. The atraumatic separation of the surrounding structures from the endoscope may increase the angle of view of the surrounding structures and may also improve the focus of the endoscope. Ports 108, 110, 112, and 114 may be configured for any of a variety of usages, including but not limited to infusion/drainage/suction of fluids or materials, insertion/removal or supporting an endoscope or fiber-optic device, inflation/deflation of the inflatable balloon 116, and for insertion/removal or support of other instruments or tools. An optional housing 118 or a handle structure may also be provided at the proximal end 104 of the shaft 102. The housing 118 may facilitate manipulation of the balloon cannula device by the user, in addition to optionally supporting the ports 108, 110, 112, and 114 and an optional steering mechanism 120 or steering assembly. The steering mechanism 120 may be manipulated using one or more actuators located on the housing 118. In the particular embodiment depicted in FIG. 1, the actuator comprises a lever 122 projecting from the housing 120, but in other embodiments, any of a variety of actuators may be provided. These and other components of the balloon cannula device 100 are described in greater detail below.

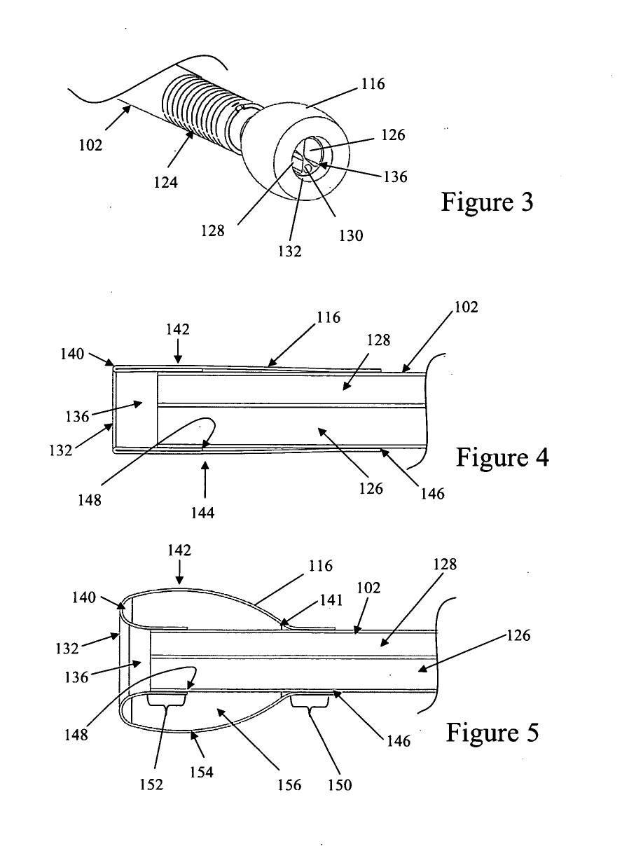

The shaft 102 of the balloon cannula device 100 may include one or more working channels. In FIG. 3, the shaft 102 is depicted with three channels 126, 128, and 130 that terminate at the distal end 106 of the shaft 102. One or more channels may have a longitudinal length that substantially spans the length of the tubular shaft 102, but other channels may have a length shorter than the tubular shaft 102, and may terminate proximal to the distal end 106. For example, FIG. 7 depicts the shaft 102 comprising an inflation/deflation channel 132 which ends proximal to the distal end 106 of the shaft 102, and may be used to control the inflation and deflation of the balloon 116. Communication between the inflation/deflation channel 132 and the balloon cavity 156 of the balloon 116 is provided by a balloon channel/cavity opening 134. Other embodiments may comprise a fewer or a greater number of channels. Other channels may also be used, for example, to control bending or other movements of the cannula device. One or more channels may comprise a layer or coating to facilitate sliding of instruments within the channel, including PTFE and any of a variety of biocompatible lubricious coating materials. In some embodiments, the shaft may comprise a rigid or semi-rigid material, but in other embodiments, may comprise a flexible material.

Proximally, one or more of the lumens or channels 126, 128, 130 and 132 of the tubular shaft 102 may be in communication with one or more ports 108, 110, 112 and 114. In the embodiment depicted in FIGS. 1 to 3, for example, one of the channels 128 of the balloon cannula device 100 is in communication with an endoscopic port 114, while another channel 126 is in communication with an instrumentation port 112, and still another channel 130 is in communication with an irrigation/aspiration port 108. In some embodiments, a separate irrigation port and aspiration port may be provided, which may permit simultaneous infusion and aspiration. Simultaneous infusion and aspiration may expedite clearing of the working field when compared to alternating infusion and aspiration using a single channel.

Distally, the visualization channel 128 may terminate about the distal end 106 of the shaft 102. The visualization channel 128 may be used as a passage for insertion/removal of illumination, visualization, and/or imaging components to provide direct visualization capabilities at the distal end 106 of the balloon cannula device 100. In some embodiments, a visualization channel 128 may house or may be integral with one or more illumination, visualization, analytical, and/or imaging components, including but not limited to one or more fiber-optic strands used to transmit light from a light source or to optically visualize the anatomy about the distal end 106 of the shaft 102.

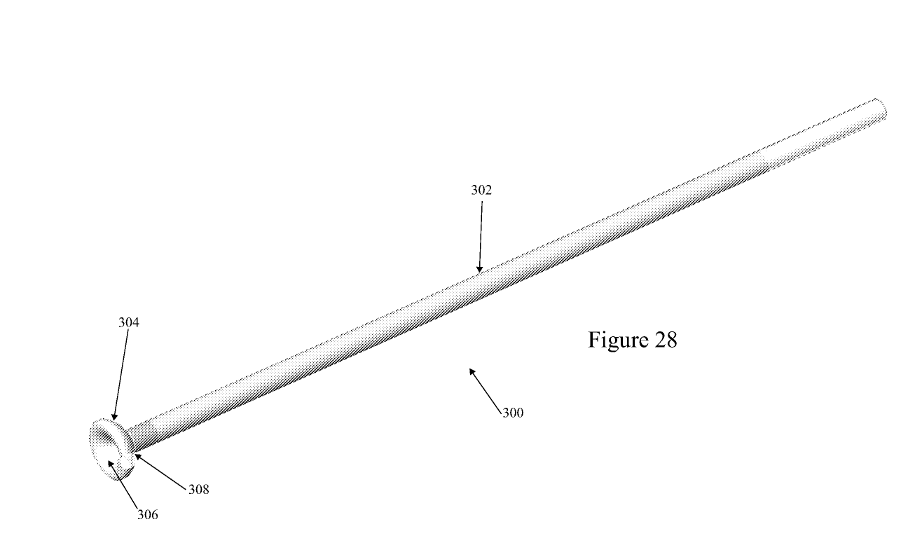

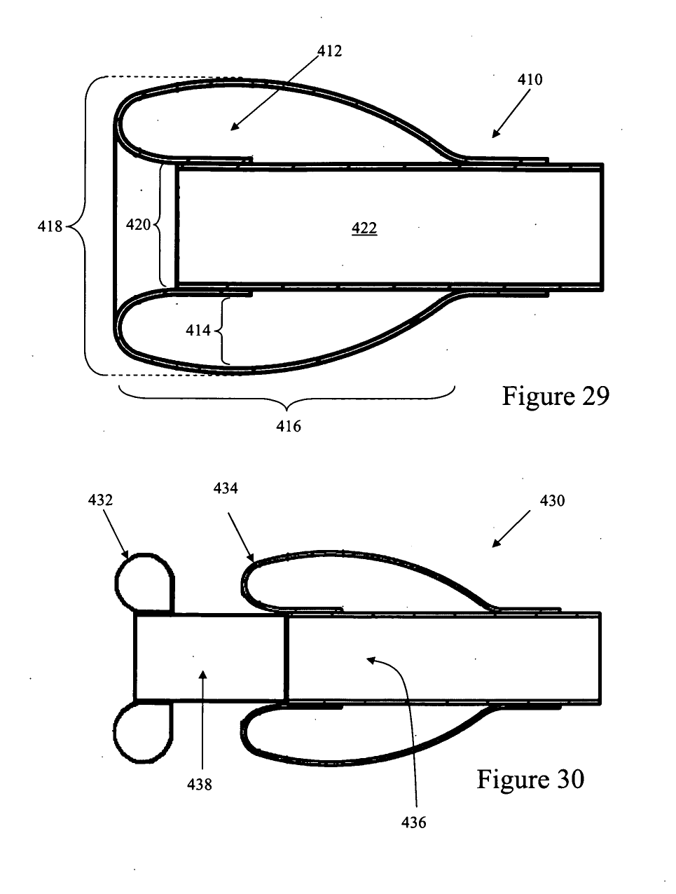

As noted in the embodiment depicted in FIG. 3, the visualization channel 128 provides access to the target area for endoscopic imaging and/or medical imaging components. In some embodiments, imaging capabilities may be augmented by one or more structures located about the distal end 106 of the tubular shaft 102. For example, a distal standoff structure may be provided to maintain some separation or spacing between the imaging device and the target region, and/or between the distal end 106 of the shaft 102 and the target region. In some embodiments, the field of view of the endoscope may be characterized by the diameter of the balloon cannula shaft plus two times the longitudinal length of the common lumen and/or balloon lumen times the tangent of half of the maximum viewing angle from endoscope and through the common balloon lumen. Thus, by increasing the distance between the endoscope and the target object, the field of view may be increased. In another example, a distal lumen segment may comprise a greater cross-sectional area, which may widen the field of view or viewing angle of the working field. In some embodiments, the field of view may be increased by increasing the length of the common balloon lumen. However, in some embodiments, increases in the common balloon lumen length may be offset by reductions in the viewing angle. This may be due to inward bulging of the balloon lumen with increases in balloon lumen length. In other embodiments, the distal end of the balloon may be configured to outwardly expand or flare upon inflation. Referring to FIG. 28, in some embodiments, the balloon 410 may be configured such that the ratio of the expanded lateral diameter 412 of the balloon lumen 414 and the length 416 of the balloon 410 is between about ½ to about 2, sometimes about ⅔ to about ¾, and other times about 0.9 to about 1.2. In some embodiments, the diameter 412 of the balloon lumen 414 may also be characterized as the total expanded diameter 418 of the balloon 410 minus the diameter 420 of the tubular shaft 422. In some embodiments, balloons with ratios less than about 0.5 may have balloon lumens that exhibit a tendency to collapse inward upon expansion, while balloons with ratios greater than about 2 may have balloon lumens that exhibit a tendency to flare or expand outwardly upon inflation.

In the particular embodiment depicted in FIG. 3, the balloon 116 comprises a balloon working lumen 136 that contains the distal end 106 of the tubular shaft 102. The balloon working lumen 132 has a greater cross-sectional area than the visualization channel 128, and in the particular embodiment in FIG. 3, provides a distal common lumen for all of the channels 126, 128 and 130 that terminate at the distal end 106 of the shaft 102. In use, an endoscopic or other type of imaging or sensing component may be positioned at with respect to the most distal opening 132 of the balloon 116. As described in greater detail below, tissue differentiating sensors or their functional equivalent may also be provided through the ports.

Embodiments of the balloon cannula device 100 may facilitate the positioning of an instrument in a targeted area. For example, the instrument may be steered using information, such imaging or physiological data, generated by a data device located on the instrument. The image may come from a data device such as a camera placed on the distal end of the instrument, or from a sensor or combination of sensors. In one embodiment, the sensor utilizes light to generate the image. In another embodiment, the sensor is adapted to see through the bloody field as presented in the spinal region by selecting at least one infrared wavelength transparent to blood or other bodily fluids. In some embodiments, at least one infrared wavelength transparent to blood presented in the spinal field may have a wavelength of about 1 micron to about 15 microns. In another embodiment, the at least one infrared wavelength transparent to blood presented in the spinal field has a wavelength between about 1.5 micron to about 6 microns. In yet another embodiment, the at least one infrared wavelength transparent to blood presented in the spinal field has a wavelength between about 6 microns to about 15 microns. In yet another embodiment, the at least one infrared wavelength transparent to blood presented in the spinal field has a wavelength between about 1.0 microns to about 1.5 microns, about 1.5 microns to about 1.9 microns, about 2.0 microns to about 2.4 microns, about 3.7 microns to about 4.3 microns, or about 4.6 microns to about 5.4 microns. In yet another embodiment, the wavelength is selected or adapted for use in distinguishing nervous tissue from surrounding tissue and/or minimally vascularized nervous tissue. In yet another embodiment, the wavelength is selected to distinguish nervous tissue from muscle. Wavelength selection information and characterization and other details related to infrared endoscopy are found in U.S. Pat. No. 6,178,346; US Patent Application Publication No. 2005/0014995, and US Patent Application Publication No. 2005/0020914, each of which is hereby incorporated by reference in its entirety for all purposes.

The visualization channel 128 or the distal end 106 of the device 100 may include a sensor used to generate images or identify tissue. In one example, the sensor utilizes acoustic energy to generate the image, similar to diagnostic ultrasound. In another example, the sensor utilizes an electrical characteristic to generate the image or other types of structural or physiological information. In yet another example, the sensor distinguishes the type of tissue adjacent to the sensor. Some properties used by the sensor to differentiate adjacent structures or tissue include resistance, capacitance, impedance, membrane voltage, acoustic, and optical characteristic of tissue adjacent the sensor or probe. Additionally, the sensor or image may be used to distinguish different types of tissue to identify neurological tissue, collagen, or portions of the annulus, for example. It is to be appreciated that the sensor may be a multi-modal or multi-sensor probe that can distinguish bone, muscle, nerve tissue, fat, etc. to help position the probe in the proper place.

In some embodiments, a trocar may be guided using fluoroscopic or other external imaging modality to place the trocar in proximity to a treatment area. In contrast to conventional procedures that attempt to fluoroscopically navigate a trocar tip around nerves and other tissue, the trocar may remain safely positioned away from sensitive structures and features. In one embodiment, the trocar tip remains about 1 to about 2 cm or more from vulnerable nerve tissue. In another embodiment, the last about 1 to about 2 cm of travel to a therapy site is performed using direct visualization provided by a visualization mechanism in the balloon cannula device.

In some embodiments, the trocar is removed and the balloon cannula device 100 is inserted into the pathway formed by the trocar. In other embodiments, a tubular trocar may be used. From the final trocar position, the balloon cannula device 100 may be passed through a channel or lumen of the trocar and along the remaining distance to the therapy or treatment site using the onboard visualization capabilities. The onboard visualization may be used alone or in combination with the balloon 116 or other type of atraumatic tip to identify, atraumatically displace, and/or maneuver around nerves and other tissue as needed. An optional steering mechanism may be provided on the balloon cannula device 100 to manipulate surrounding tissue and structures, and/or to traverse the remaining distance to one or more therapy or treatment sites. In other embodiments, the balloon cannula device 100 may have a rigid or fixed configuration, and may be manipulated by optionally manipulating the trocar to reach a desired location. In an alternative embodiment, the trocar may house the balloon cannula device during trocar insertion and thus utilize the direct visualization capabilities of the visualization mechanism within the balloon cannula device to guide trocar positioning. In still another embodiment, the trocar may be provided with a separate imaging system from the imaging device or component provided in the balloon cannula device for use during trocar insertion. In still another embodiment, the trocar may be configured with a lumen to house only the imaging component from the balloon cannula device 100. After the desired trocar position is reached, the trocar is removed and the imaging component is removed from the trocar and reinserted into the balloon cannula device 100. In yet another alternative embodiment, both external imaging may be used to position the trocar distal end, either alone or in combination with direct imaging.

As mentioned previously, one or more embodiments of the balloon cannula device may be provided with any of a wide variety of steering configurations, such as the steering mechanism 120 depicted in FIG. 1. In one embodiment, the balloon cannula device 100 is steerable in one or more axes, including a device with two axes. In some embodiments, one axis may be a rotation axis. In another embodiment, the balloon cannula device is non-steerable. In yet another alternative embodiment, the balloon cannula device may be pre-formed into a shape that is adapted to access a portion of the spinal region or other region of the body. The shape may include any of a variety of angled and/or curved segments to access a particular body site. In yet another embodiment, the balloon cannula device is situated within the trocar in such a way that the balloon cannula may have steering capability up to about 360° inside the spinal space. The steering mechanism 120 may include one or more flexible bodies or flex regions 124 on the balloon cannula device 100. The flexible body may be bent by manipulating a control such as lever 122 located on the housing 118. Various examples of the steering mechanism and the flex region 124 and are described in greater detail below.

The dimensions of the balloon cannula device may be sized and selected based on the particular therapy being provided. For example, one embodiment of the balloon cannula device may be dimensioned for navigation to a spinal region for diagnostic evaluation and/or to apply a therapy thereto. In another embodiment, the balloon cannula device may be sized to fit within the epidural space. Other embodiments may be configured for use in the chest cavity (e.g. pleural biopsy or pleuracentesis) or abdominal-pelvic cavity (e.g. bladder neck suspension), or for non-spinal procedures such as breast biopsy and transvaginal oocyte retrieval, for example. In some embodiments, the balloon cannula device 100 may have a diameter of about 5 mm or less, while in other embodiments, the diameter may have a diameter of about 3 mm or less, or even 2.5 mm or less. In another embodiment, one or more of the working channels 124, 126 and 128 of the balloon cannula device 100 may have a diameter of about 5 mm or less, about 3 mm or less, about 2 mm or less, about 1 mm or less, or about 0.8 mm or less.

As mentioned previously, the cannula device may comprise a balloon or other type of structure that may be used as an atraumatic tip structure to reduce the risk of inadvertent injury to surrounding structures during a procedure. The atraumatic tip may be configured to provide tactile feedback to the user of the rigidity, pliability or feel of the tissue or structures in contact with the tip. In one embodiment, the atraumatic tip also provides dissection or retraction capabilities and/or the ability to displace surrounding tissue. The overall shape of the atraumatic tip may allow manipulation of the nerves as the balloon cannula device is advanced without harming the nerve or causing pain. In one embodiment, the atraumatic tip may have a curved shape and no sharp edges, burrs or other features that may pierce, snag, tear or otherwise harm tissue that comes into contact with the atraumatic tip. The shape, surface contours and/or overall finish of the atraumatic tip may be selected to reduce or minimize impact forces when the tip comes into contact with structures such as nerves, muscle and the spinal dura, among others.

As mentioned previously, the atraumatic tip may also be controllably inflatable or expandable, or otherwise comprise two or more surfaces or structures that are independently controllable. One potential use of such an embodiment comprises contacting the tip against tissue and then inflating, expanding or separating to deform or move the tissue. The tip may be a balloon that is inflated to create a working space in the surrounding tissue as well as provide a clearing for improved visibility. The balloon cannula device may then be advanced into the working space. The balloon may be inflated again to create another working space and so forth to advance the balloon cannula device in a spinal space. In addition, the balloon cannula device may be used to provide saline or another type of cleaning solution to the working area for enhancing visualization. In another embodiment, the distal balloon 116 may be moveable or articulated such that it may be used to displace, nudge or prod surrounding tissue or structures. The nudge action is felt by the user and also provides a more tactile sense of tissue movement. The nudge may result from active movement of the tip under control of the user, movement caused by releasing the tip from a bias position or from other conventional techniques for manipulation of surgical implements.

FIGS. 4 and 5 illustrate the balloon 116 of the balloon cannula device 100 in the stowed condition and deployed condition, respectively. As shown, a portion of the distal end 140 of the balloon 116 is located distal to the distal end or tip 106 of the tubular shaft 102. In one embodiment, the balloon 116 is distally located about 5 mm or less past the shaft tip 106, sometimes about 3 mm or less past the shaft tip 106; other times about 2 mm or less past the shaft tip 106. The net longitudinal length of the balloon 116, as mounted on the tubular shaft 102, may be in the range of about 3 mm to about 20 mm, sometimes about 4 mm to about 10 mm, and other times about 5 mm to about 8 mm, for example. In one embodiment, the distal end 140 of the balloon 116 is located about 1 mm to about 1.3 mm In the uninflated state, the balloon 116 may have an outer diameter of about 4 mm or less, sometimes about 3.6 mm or less, and other times about 3 mm or less. In the inflated state, the balloon 116 may have a maximum outer diameter of about 4 mm or more, sometimes about 5 mm or more, and other times about 6 mm or more. The outer diameter of the balloon 116 may vary, depending upon on the particular balloon configuration and the degree of inflation. In some embodiments, the balloon cannula device 100 may be configured to withstand inflation pressures up to about 60 psi or less, while in other embodiments, the balloon cannula device 100 may be configured to withstand inflation pressures up to about 80 psi or less, or sometimes about 100 psi or less, and other times up to about 200 psi or more. In some particular embodiments, the balloon cannula device 100 may be configured to provide a diameter change of about 2 mm or more across a pressure change of about 50 psi (e.g. from about 30 psi to about 80 psi), sometimes about 2.5 mm or more, and other times about 3 mm or about 4 mm or more. In some embodiments, the balloon 116 may be configured such that the peak ratio of diameter change to pressure change occurs in the range of about 30 psi to about 120 psi, sometimes about 40 psi to about 100 psi, and other times about 60 psi to about 80 psi. In some embodiments, inflation of the balloon cannula device 100 to a pressure of at least about 45 psi or more may reduce the degree of balloon 116 deformation during use, which may improve the tactile feedback of the balloon cannula device 100. While balloons inflated to semi-rigid or rigid pressures may exhibit less deformation upon contact with structures such as nerves, the shape of the balloon 116 with a curved distal tip and a tapered proximal end may also provide the balloon 116 with a shape that by atraumatically displaces away such structures upon contact.

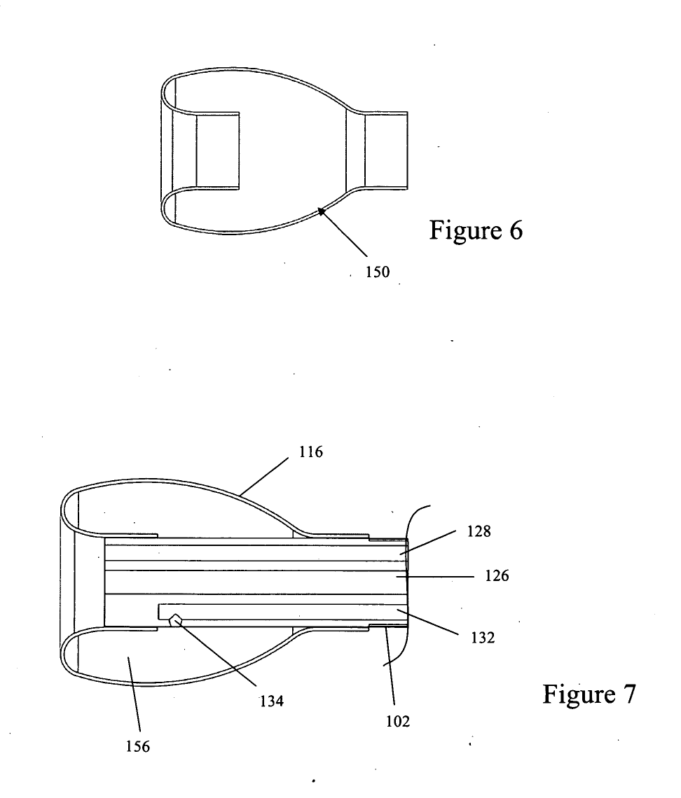

In some embodiments, the balloon component may be formed such that its base configuration is an uninflated shape, an inflated shape, or an intermediate shape therebetween. In some embodiments, a balloon with its base configuration in the uninflated shape may lie flatter against the tubular shaft of the balloon cannula device in comparison to a balloon with its base configuration in the inflated shape. In other embodiments, balloons having a base configuration in their inflated shape may have ripples, wrinkles or folds when collapses or constricted for insertion into the body. In other embodiments, such as the balloon 160 in FIG. 6, balloons having a base configuration in their inflated state may have more controllable or predictable conformation in their inflated state, when compared to balloons having a base configuration in their uninflated state. In the particular embodiment depicted in FIGS. 4 and 5, the balloon 116 comprises a tubular structure 144 having a proximal end 146 and a distal end 148. The material comprising the tubular structure 144 may have a uniform or non-uniform thickness, and a uniform or non-uniform axial cross-sectional area or shape. The proximal end 146 is attached at a proximal mounting site 150 that is proximal to the distal end 106 of the tubular shaft 102, while the distal end 148 is attached at a distal mounting site 152 and is folded under a middle portion 154 of the balloon 116 such that at least a portion of the middle portion 154 is located distal to the distal end 106 of the tubular shaft 102. As may be seen in FIGS. 4 and 5, the tubular structure 144 may be characterized as an inverted or everted configuration, where the inner surface of the tubular structure is attached to the proximal mounting site 150 while the opposite or outer surface of the tubular structure is attached to the distal mounting site 152. During manufacture of the balloon cannula device, an inner diameter of one end of the tubular structure 144 may be bonded to the distal end 106 of the tubular shaft 102, while the other end of the tubular structure 144 is free and distal to the distal end 106 of the shaft 102. The free end of the tubular structure 144 may then be everted and pulled back over the attached end of the tubular structure 144 and attached to the tubular shaft 102 proximal to the attached end of the tubular structure 144. The proximal attachment site may be selected so that at least a portion of the tubular structure 144 is distal to the distal end 106 of the tubular shaft 102. Alternatively, the proximal attachment of the tube 144 may be made and then the distal end of the tube 144 may be inverted and attached to the shaft 102.

Although both the proximal and distal mounting sites 150 and 152 may be located on the same tubular shaft 102, in some embodiments the mounting sites 150 and 152 may be located on different tubular shafts have a coaxial sliding relationship. In this latter embodiment, the two tubular shafts may be manipulated to alter the balloon shape. For example, the proximal and distal mounting sites 150 and 152 may be brought closer together to permit a greater radial expansion range. In another example, the proximal mounting site 150 may be moved more proximally, which in some embodiments, may shift the balloon proximally to elongate the balloon configuration and/or to reduce the degree of forward positioning of the balloon.

In some embodiments, the balloon or tubular structure may be attached to the tubular shaft by adhesives or by heat bonding, for example. In some embodiments, bonding structures or processes may be used, which may improve the sealing between the balloon and the tubular shaft to support the use of higher inflation pressures without separating the balloon from the tubular shaft. For example, crimp rings or heat shrink tubing may be used to augment the bonding or attachment process. In some embodiments, the crimp rings or shrink tubing may be applied temporarily to facilitate setting of other bonding processes, and are later removed. In other embodiments, the crimp rings or shrink tubing may be incorporated into the final assembled product.

In some embodiments, after inflation, the balloon may not fully revert to its uninflated state upon relief the inflation pressure. Due to stretching or other types of deformation, the balloon may fold, wrinkle or crease upon deflation, which may affect the ability to withdraw the balloon cannula device from an introducer or guide into which the balloon cannula device was inserted. In some embodiments, where the balloon material comprises a polymeric material, the deflation characteristics of the balloon may be improved providing at least some polymer chains that are circumferentially oriented with respect to the tubular shaft. In embodiments where the balloon 116 originates as a thermoplastic tube, the thermoplastic tube may be an extruded polymeric tube, which typically provides longitudinally oriented polymer chains due to the extrusion process used to form the tube. In some embodiments, some of the longitudinally oriented polymer chains may be reoriented toward a circumferential orientation by heating the tube while in an expanded state.

In one example, a thermoplastic tube is provided, comprising an outer diameter that is less then the final assembled diameter of the balloon, and a thickness that is greater than the final thickness of the balloon. The thermoplastic tube is placed into a molding tube or cavity having an inner diameter that is approximate to the final assembled balloon outer diameter. The thermoplastic tube is pressured and expanded until the outer surface of the tube is constrained by the inner diameter of the modeling tube or cavity for a period of time at an elevated temperature. The thermoplastic tube is then cooled while pressurized to set the new diameter and to set the circumferentially reoriented polymer chains. The temperature, pressure and treatment period of the thermoplastic material may vary, depending upon the particular balloon material and the balloon configuration. In one particular example wherein the thermoplastic material comprises polyurethane with a durometer in the range of about 80 A to about 95 A and a thickness of about 0.005″ to about 0.01″, sometimes about 0.006″ to about 0.009″, and other times about 0.007″ to about 0.008″. The setting temperature may be about 120° F. to about 250° F., depending upon the particular material used. The setting period may be in the range of about 5 seconds to about 2 hours or more, sometimes about 30 seconds to about 30 minutes, and other times about 1 minute to about 2 minutes.

The balloon may be made of a flexible material such that the balloon is inflatable upon introduction of a fluid or gas. In one embodiment, the flexible material has sufficient rigidity such that it may effectively maintain the tubular shape when uninflated. As shown in FIG. 5, the distal end 140 of the balloon 116 may remain extended past the distal end 106 of the shaft 102 before and after inflation. Embodiments of the atraumatic balloon 116 may also be used to assist with or perform therapy or treatment, shield surrounding tissue or provide access for other devices. The atraumatic balloon 116 may be positioned at the surgical or treatment site in a compact or stowed condition (see, e.g., FIG. 4) and then deployed according to the type of device used (see e.g., FIG. 5).

The atraumatic balloon 116 may be used to manipulate surrounding tissue in one or more ways. First, by transitioning the balloon 116 from a stowed to deployed configuration, the outer walls 142 of the balloon 116 will be urged outward against the surrounding tissue. Second, whether or not the device 100 is deployed or stowed, the balloon cannula device 100 may be maneuvered using the steering mechanism 120 to manipulate tissue. Third, the atraumatic balloon 116 may cycled between the stowed and deployed configuration to assist in the advancement of the steerable balloon cannula device 100. For example, the balloon 116 may be deflated to facilitate insertion of the device 100 through a wall of tissue, and may be reinflated after traversing the wall. Fourth, the practitioner may advance the balloon cannula device 100 and manipulate surrounding tissue and push tissue away by creating space under direct visualization. As the balloon 116 of the balloon cannula device 100 expands, a work space or opening may be created in the surrounding tissue, thereby easing the advancement or atraumatic maneuverability of the balloon cannula device 100. Thereafter, the atraumatic balloon 116 may be deployed or otherwise used to deform surrounding tissue and/or to make space available for the balloon cannula device 100 or other therapy or treatment device provided by working channel 126 (e.g., FIG. 3). It is contemplated that one or more of these methods may be used in combination to manipulate the surrounding tissue. Any of a variety of other methods for utilizing the balloon cannula device 100 are also contemplated.

In the embodiment illustrated in FIG. 7, the atraumatic balloon 116 is an inflatable structure. The balloon 116 may be adapted for delivery via trocar or introducer. As shown in FIG. 4, in one embodiment, the balloon 116 may be folded, compressed or stowed in such a manner that the balloon 116 is deliverable with an embodiment of the balloon cannula device via the trocar or introducer. Additionally, the balloon 116 may be held by a sheath to retain expandable structures in a constrained configuration. Once the balloon 116 is positioned where desired, the sheath may be removed to allow the device to transition to a deployed configuration.

Exemplary embodiments of the tip structure(s) may include any of a variety of balloons or other shaped inflatable structures. There are a great many different shapes, sizes and functionality readily available in such balloons and many are well suited and easily adaptable for use in endoscopic spinal procedures. In one embodiment, the balloon, when in a stowed configuration, is dimensioned to translate through a lumen, working channel, trocar or introducer in an embodiment of the balloon cannula device described herein. The atraumatic balloon may be shaped in virtually any shape desired to further balloon cannula. For example, the balloon may be elongated, rounded, or other pre-formed shape. In one specific embodiment, the balloon has an elongate shape that follows the shape of an adjacent spinal structure. In one specific embodiment, the balloon is adapted to follow a portion of the dura mater. In another specific embodiment, the balloon is adapted to follow a portion of the annulus. In another embodiment, the atraumatic balloon includes a marker or other feature(s) making all or a portion of the balloon perceptible using external imaging modalities. In another embodiment, the atraumatic balloon is donut shaped to create a forward-looking design. In one embodiment, the marker or feature is a radio opaque marker. In other embodiments, including the balloon cannula device 430 in FIG. 30, multiple balloons 432 and 434 may be provided. The multiple balloons may be arranged serially along the balloon cannula device 430, and/or in a parallel fashion (e.g. two semi-cylindrical or semi-tubular balloons located at the distal end of a balloon cannula system). The multiple balloons need not have the same size and/or configuration. Embodiments comprising multiple balloons may comprise independently controllable balloons, or balloons that inflate in a coordinated fashion.

Atraumatic balloon embodiments are not limited to closed surface, inflatable embodiments. Open surface structures such as mesh, scaffold structures, polymer stent-like structures, for example, may also be used to atraumatically deform spinal tissues. One example of an open surface structure is a coronary stent. Many of the delivery techniques used to deliver stents into the vasculature are applicable here for delivery into the spinal space. The stent may also be a polymer stent or a stent with a coating to improve the atraumatic qualities of the stent to spinal tissues and structures. In another embodiment, a suitable scaffold includes the collapsible scaffold structures used to deform and support tissue, and to maintain spacing between a radioactive source and the tissue being treated prior to and during brachytherapy.

In one embodiment, the surfaces of the atraumatic balloon are expandable. For example, the atraumatic balloon might be expanded using mechanical mechanisms, pneumatic mechanisms, or hydraulic mechanisms. In addition, the atraumatic balloon 116 may also contain sensing and/or monitoring devices such as a temperature thermo-couple. In an alternative embodiment, the atraumatic balloon may include multiple layers and provide insulation or shielding to surrounding tissue by changing thermal and/or insulating properties either alone or in combination with expansion and contraction between the multiple layers. The change in properties could be accomplished by electrical, chemical, or mechanical properties of the layers, spaces between layers or through the use of a liquid, gas or other material inserted between layers or into a layer.

In some embodiments, the atraumatic balloon may not be circular in cross section or may not be generally cylindrical as the balloons suited for use in the vasculature. In one embodiment, the atraumatic balloon may be irregular in shape and may be designed to accommodate tissues, endoscopes, and therapeutic devices to avoid potential injury to the tissues during treatment options. In one embodiment, the atraumatic balloon is adapted to conform to a portion of the spinal anatomy when in a deployed configuration. In another embodiment, the atraumatic balloon is sized and adapted to conform to the shape of the annulus. In another specific embodiment, the atraumatic balloon may have a preformed shape, such as a rounded shape, an elongated shape or combinations thereof. In one specific embodiment, the folded wall thickness of the atraumatic balloon is about 6 to about 40 thousandths of an inch. In another specific embodiment, the folded wall thickness of the atraumatic balloon may be about 12 to about 30 thousandths of an inch. In another specific embodiment, the folded wall thickness of the atraumatic balloon is about 20 to about 30 thousandths of an inch. Other sizes are possible and may be selected based on the channel size of the balloon cannula device as well as the physical parameters of the patient's spinal area.

FIG. 7 is another cross-sectional view of the atraumatic balloon 116 depicting the structures relating to the inflation of the balloon 116. The atraumatic balloon 116 not only provides the capabilities of the balloon 116 but may also include working channels to further assist in performing procedures. The balloon 116 is capable of both stowed and deployed configurations and is illustrated in a deployed configuration in FIGS. 4 and 5. The access lumens 126, 128, and 130 may run the length of the device 100 and may be sized to allow passage of the catheters, endoscopes, and instruments/devices, respectively. The balloon lumen 132 may be adapted to inflate the balloon 116. As shown, the distal portion of the lumen 132 may include a port 134 in fluid communication with the balloon 116. The balloon 116 may be filled with contrast solution in order to improve fluoroscopic visualization of the device balloon or device. In other embodiments, the balloon may not require an inflation/deflation port 132. Instead, the balloon cavity may optionally be filled with compressible or non-compressible gas or liquid, which may be redistributed or compressed out of the balloon using a sheath or other constraining structure.

In some embodiments wherein the atraumatic tip comprises a balloon structure, the balloon structure may be inflated with an optically transparent fluid that is selected to reduce distortions through the balloon and/or cavity, and/or reflections at the balloon/cavity interface. An optional bubble removal filter may be provided with the cannula system or the fluid injection system to reduce the number of bubbles inflated into the balloon. In some embodiments, visibility through the balloon structure may be different between the uninflated state and inflated state. In some embodiments, where the curvature of the inflated balloon may reduce the visual clarity through the balloon, the balloon may be configured to limit the amount of curvature or expansion in one or more areas. In FIGS. 11A and 11B, for example, the distal segment 200 of a forward-mounted balloon 202 may comprise two or more attached, fused or adhered balloon surfaces 204 and 206 so as to resist expansion or separation. These embodiments may improve visual clarity through the distal segment 200 while providing an expandable segment 208. In other embodiments, the balloon may be configured to permit at least some inflation without substantially changing the relative spacing between the two balloon surfaces, e.g. where the balloon surfaces remain longitudinally straight and parallel. Such embodiments may permit distal expansion while maintaining some visualization through the distal segment of the balloon in its expanded state. The distal segment 200 of the forward-mounted balloon 202 may have a length that is smaller, greater or similar to the forward balloon length 210 that is distal to the distal end 212 of the tubular shaft 214.

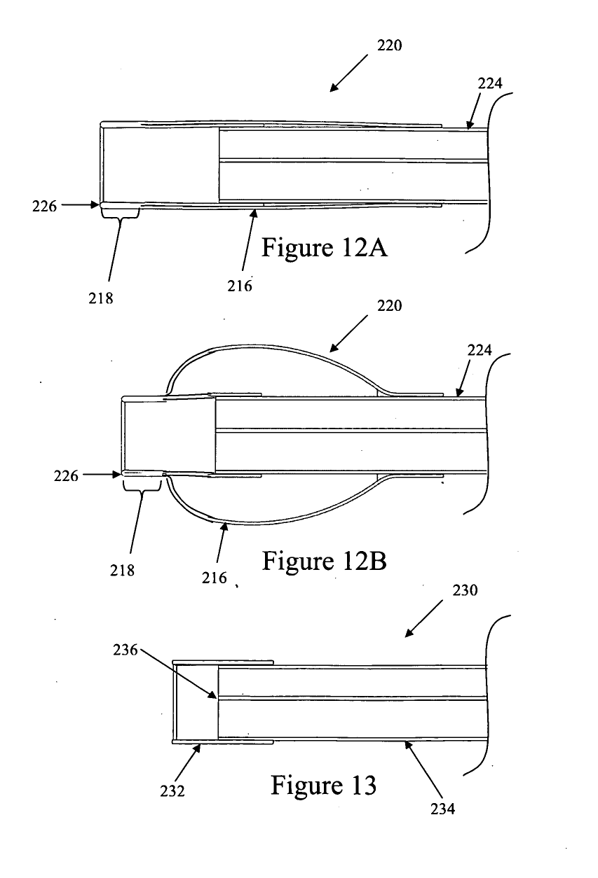

Although the embodiment depicted in FIGS. 11A and 11B comprises a bi-layer distal segment 200, as illustrated in FIGS. 12A and 12B, for example, a forward-mounted balloon 216 with a single-layer distal segment 218 may be provided. A single-layer distal segment 218 may further augment visual clarity by eliminating one interface between the layers, inflation fluids, and/or the use of any adhesives, if any. The thickness of the single-layer distal segment 218 may or may not be about equal to the thickness of a layer comprising the expandable segment. In other embodiments, for example, the distal segment may comprise three or more layers. The average thickness of the distal segment may vary from about 10 microns to about 500 microns or more, sometimes about 11 microns to about 200 microns, and other times about 12 microns to about 150 microns. In some embodiments, the non-expandable distal segment may have a longitudinal length of about 1 mm to about 10 mm or more, sometimes about 2 mm to about 8 mm, and other times about 4 mm to about 6 mm.

In some embodiments, the distal segment may comprise a different material from the proximal balloon material. The distal segment may be attached to the balloon in any of a variety of ways, including but not limited to the use of adhesives or heat bonding. In other embodiments, such as the cannula device 230 depicted in FIG. 13, a non-inflatable atraumatic tip 232 may be attached directly to the tubular shaft 234 in a forward position. Here, no expandable segment is provided, but in other embodiments, a non-forward mounted expandable segment may be provided on the tubular shaft 234, in addition to the non-inflatable atraumatic tip 232. The non-forward mounted may be flush mounted or proximally mounted with respect to the distal end 236 of the tubular shaft 234.

The distal segment or tip 232 in FIG. 13 may be selected from a material that is transparent to the operation of the port components, such as visualization channel. The atraumatic tip 232 maintains a working space about the distal end 238 about the visualization channel 240 while leaving the other working channels open for the introduction of instruments and/or endoscopes. In some embodiments, the atraumatic tip 232 may be formed from rigid, clear plastic, while in other embodiments, the atraumatic tip may comprise a flexible, deformable material. In some embodiments, the tip comprises an opaque material, but in other embodiments may be translucent or transparent, which may facilitate the visualization of the tissue or structures adjacent the tip. The tip material may be stainless steel, cobalt chromium, titanium, nickel-titanium, polycarbonate, acrylic, nylon, PEEK, PEK, PEKK, PEKEK, PEI, PES, FEP, PTFE, polyurethane, polyester, polyethylene, polyolefin, polypropylene, glass, diamond, quartz, or combination thereof, for example. In some embodiments, the tip materials may include the addition of one or more radiographic markers or materials.

In other embodiments, the forward-mounted balloon or expandable structure may be provided with one or more support elements. The support elements may be oriented longitudinally, radially, and/or circumferentially along the balloon to support the uninflated and/or inflated balloon configurations. The configuration of a support element may be complementary to the shape or configuration of the balloon. In one embodiment, the support element may comprise a helical configuration, for example. In some embodiments, the support elements may be located about the balloon lumen. Support elements about the balloon lumen may resist involution of the balloon lumen in the inflated configuration, particularly at higher inflation pressures. The support elements may comprise any of a variety of materials, including but not limited to a metal and/or polymeric material. The support element maybe rigid, semi-rigid or flexible, and at least a portion of the support element may be attached or coupled to the shaft, the inner or outer surface of the balloon, and/or embedded in the balloon wall.

Although the balloon 116 may be generally circumferentially symmetrical about the longitudinal axis of the shaft 102, in other embodiments, the balloon may be asymmetrical. In FIG. 5, the balloon 116 comprises a toroidal configuration wherein the distal end 140 of the balloon 156 has a curved or rounded configuration when inflated and the proximal end 141 of the balloon cavity 156 has a tapered configuration. Other balloon configurations may also be used, and slits or windows may be optionally provided to increase visualization. For example, the balloon configuration may be altered using different balloon shapes, variable wall thickness and/or by pre-forming curves or fold along one or more regions of the balloon material.

In some embodiments, the balloon may have semi-circular, hood-like configuration that is open along at least one perimeter of the balloon working space. FIG. 28 illustrates an alternate embodiment of a balloon cannula device 300, comprising a shaft 302 and a conical balloon 304 with a conical balloon working space 306 that opens to the surrounding structures. In some embodiments, the conical balloon 306 may be configured to extend and retract into the distal end 308 of the shaft 302 while expanding and contract its diameter, respectively. In other embodiments, the conical balloon 304 and/or conical balloon working space 306 may controllably expand or contract without requiring extension or retraction of the balloon 304. This may be performed by providing two or more separately inflatable/deflatable compartments with respect to the balloon to provide non-uniform inflation forces within the balloon. In other embodiments, contractile electropolymers in or on the balloon which may be manipulated by applying a current or voltage using wires and electrodes attached or contacting the electropolymers.

FIG. 30 depicts still another embodiment of a balloon cannula device 430, comprising multiple balloons 432 and 434. In this embodiment, one balloon 432 may be located distally on the cannula 436 while a second balloon 434 is located more proximally on the cannula 436. The cannula segment 438 between the two balloons 432 and 434 may be a single-lumen, transparent material. This may permit an endoscope or other direct visualization component to view the structures adjacent to the cannula segment 438 while the two balloons 432 and 434 provide some separation between the structures and the cannula segment, which may broaden the viewing angle.

Referring back to FIG. 3, the tubular shaft of the balloon cannula device 100 may include a visualization channel 128, a larger working channel 126, and an additional irrigation/aspiration port 130. The channels and/or ports of the balloon cannula device 100 may be configured to accept wide variety of therapy devices suited to the type of therapy being performed. The therapy device may be configured and used to apply energy to surrounding tissue. The therapy device may also be a surgical instrument used to cut, pierce or remove tissue. Moreover, it is to be appreciated that the therapy device may be any conventional endoscopic instrument. The therapy device may include ultrasonic devices, motor driven devices, laser-based devices, RF energy devices, thermal energy devices, cryotherapy-based devices, or other devices selected based on the spinal therapy being performed. For example, the therapy device may also be a mechanical device adapted to remove tissue such as a debrider or an aspirator. Other examples are described in greater detail below. Moreover, it is to be appreciated that the balloon cannula device 100 may be used to inject pharmacological agents into the spinal area. The size, number and arrangement of the working channels are readily adaptable for different configurations, depending upon the type of procedures performed. A greater or a fewer number of working channels may be provided, and the working channels need not have the same size and shape. In addition, the working channels may also be configured to perform auxiliary functions. In one example the channels or ports may be used to provide irrigation to assist in tissue dissection as the atraumatic tip is advanced in the spinal space. An irrigating working channel may be in communication proximally with a fluid source, such as a syringe or intravenous infusion system, and in communication distally with the distal end of the balloon cannula device so that the fluid exiting the irrigation working channel is directed to the distal portion of the balloon cannula device. In another example, the irrigation working channel or another working channel may be used to rinse the atraumatic tip or keep clear other portions of the balloon cannula tool. In the particular embodiment depicted in FIG. 3, the working channel 126 and the visualization port 128 are configured with non-circular cross-sectional shapes. In some embodiments, the non-circular shape permits the placement of an instrument with a circular cross-sectional shape within the channel or port while providing still providing flow paths for fluids and material through the channel 126 and the port 128. Shared or eccentric flow paths along non-circular shaft channels and ports may also otherwise take advantage of unused sections of the cannula shaft. Unlike shafts with only circular channels or ports, the flow paths may be provided without having to increase the overall cross-sectional area of the cannula shaft. Channels or ports having non-circular cross-sectional shapes may also be used with instruments having a complementary non-circular cross-sectional shape. For example, complementary non-circular cross-sectional shapes may be used to control or limit the amount of instrumentation rotation within the channel or port.

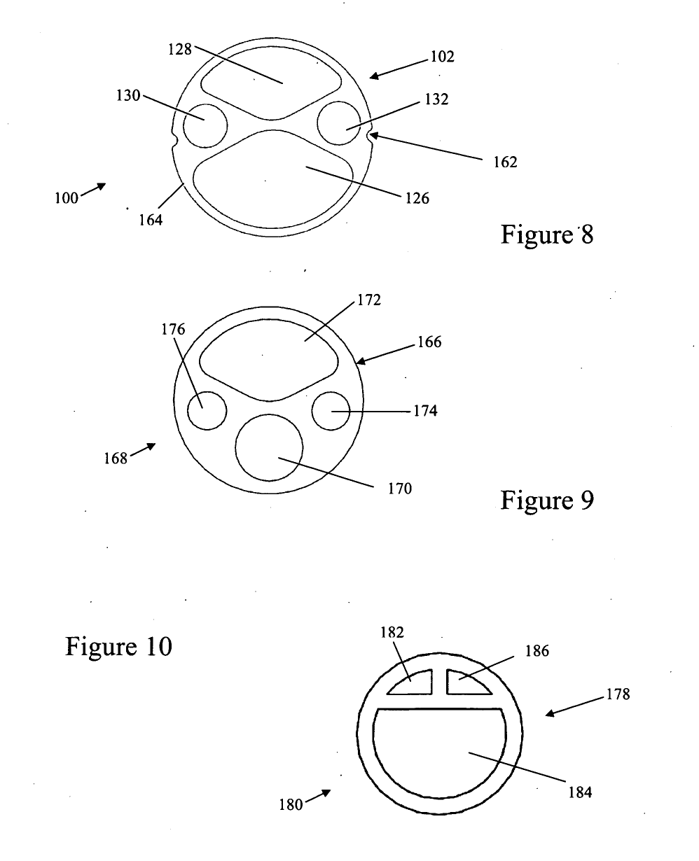

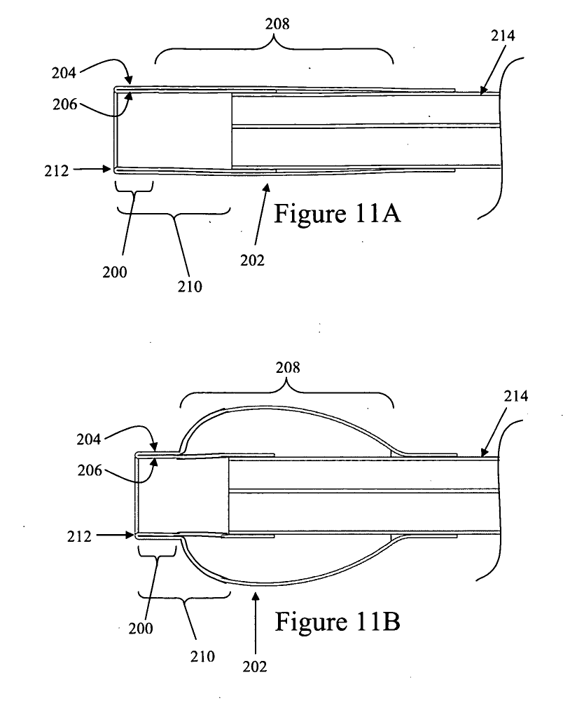

FIGS. 8, 9, and 10 illustrate various embodiments of the balloon cannula device. As shown in FIG. 8, the balloon cannula device 100 may comprise a shaft 102 with a non-circular visualization or irrigation channel 128, a non-circular working channel 126 which may be used to provide therapy device or as aspiration port, a balloon inflation lumen 132, and additional port 130 for irrigation or aspiration. The shaft 102 also optionally comprises one or more structures 162 on its outer surface 164. These structures 162 may comprise recessed or protruding configurations and may be used, for example, to maintain alignment with respect to introducer or guide member, or to reduce the amount of frictional resistance from any manipulation of the balloon cannula device 100. As depicted in FIG. 9, the shaft 166 of the balloon cannula device 168 may have a non-circular visualization or irrigation port 170, a circular therapy device or aspiration port 172, a circular balloon inflation lumen 174, and additional circular port 176 for additional irrigation or additional aspiration having a greater. As demonstrated in FIG. 9, the circular ports 172, 174 and 176 need not have the same diameter. In FIG. 10, the shaft 178 of the balloon cannula device 180 has a visualization or irrigation port 182, an injection port or therapy device or aspiration port 184, and a balloon inflation lumen 186, wherein no port or lumen has a circular cross-sectional shape. It is contemplated that functions of various lumens in a cannula device may be suitably interchanged.

During use, the balloon cannula device may be moved or may remain in place while an inserted therapy device is manipulated to perform the desired function. Once the working or therapy area has been created or accessed using the atraumatic balloon, the atraumatic balloon may be removed thereby allowing working channel or trocar or introducer to be used for another instrument or therapy device or to provide support for a procedure. For example, the therapy device may comprise a mechanical debrider or other type of tissue disrupting device that may be introduced via the working channel to assist in removal of tissue. Various examples of mechanical tissue disrupting devices that may be used with a balloon cannula device are described in U.S. patent Ser. No. 12/035,323, filed Feb. 21, 2008, which was previously by incorporated by reference in its entirety. In yet another example of the flexibility of the balloon cannula device, one or more the working channels or ports may be used to provide access for the delivery of pharmacological agents to the access site either for application onto or injection into tissue. In some embodiments, the therapeutic agents may be directed injected into the channel or port, but in other embodiments, an infusion catheter may be inserted into a channel or port and used to provide additional control of the therapy. The infusion catheter may have any of a variety of configurations and features, including but not limited to its own optional steering mechanism separate from the balloon cannula device, and a needle tip for injecting therapeutic agents into the tissues or structures. In some embodiments, the needle tip may be retractable and extendable to protect against inadvertent puncture of the balloon cannula device and/or the tissues or structures accessible from the balloon cannula device. Examples of injection catheters that may be used with embodiments of the balloon cannula device include U.S. patent Ser. No. 10/820,183, which is hereby incorporated by reference in its entirety.

The therapy device may be supplied with energy from a source external using a suitable transmission mode. For example, laser energy may be generated external to the body and then transmitted by optical fibers for delivery via an appropriate therapy device. Alternately, the therapy device may generate or convert energy at the therapy site, for example electric current from an external source carried to a resistive heating element within the therapy device. If energy is supplied to the therapy device, transmission of energy may be through any energy transmission means, such as wire, lumen, thermal conductor, or fiber-optic strand. Additionally, the therapy device may deliver electromagnetic energy, including but not limited to radio waves, microwaves, infrared light, visible light, and ultraviolet light. The electromagnetic energy may be in incoherent or laser form. The energy in laser form may be collimated or defocused. The energy delivered to a disc may also be electric current, ultrasound waves, or thermal energy from a heating element. Moreover, it is to be appreciated that embodiments of the balloon cannula devices described herein may also be used to dispense a compound, compounds or other pharmacological agents to reduce, diminish or minimize epidural neural tissue scarring.

The balloon cannula device may also be used to perform denervation procedures using direct visualization from the balloon cannula device. The denervation procedure may be physical, chemical or electrical denervation, for example. The approaches used may be similar those described herein to access the posterior or posterolateral annulus. It is to be appreciated that the denervation procedures may be performed to relieve discogenic pain and/or before the disc damage has progressed to a herniated disc or torn annulus.

Referring back to FIG. 1, as noted previously this embodiment of a balloon cannula device 100 further comprises a steering mechanism 120. During use, the balloon cannula device 100 may be advanced through the working channel of a trocar or introducer and into the working area. In some embodiments, the working area or space may be created by separating structures or tissue using the atraumatic balloon 116, either alone or in combination with the steering mechanism 120. The steering mechanism 120 may be configured to provide any of a variety of steering features, including various bending planes, various bending ranges, extension and retraction ranges, and rotations ranges, for example. As mentioned previously, in the embodiment depicted in FIG. 1, the actuator comprises a lever 122 with both ends 188 projecting from the housing 118, but in other embodiments, any of a variety of actuators and actuator configurations may be used, including but not limited to dials, knobs, sliders, buttons and the like, as well as electronic touch controls, for example. In some embodiments, only one end 188 of the lever 122 may project from the housing 118. The controls used to manipulate the steering mechanism 120 may be manually manipulated by the user or by a mechanical control system comprising various motors. In still other embodiments, actuators such as the lever 122 may be omitted and the balloon cannula device 100 may be directly coupled to a motor control system.

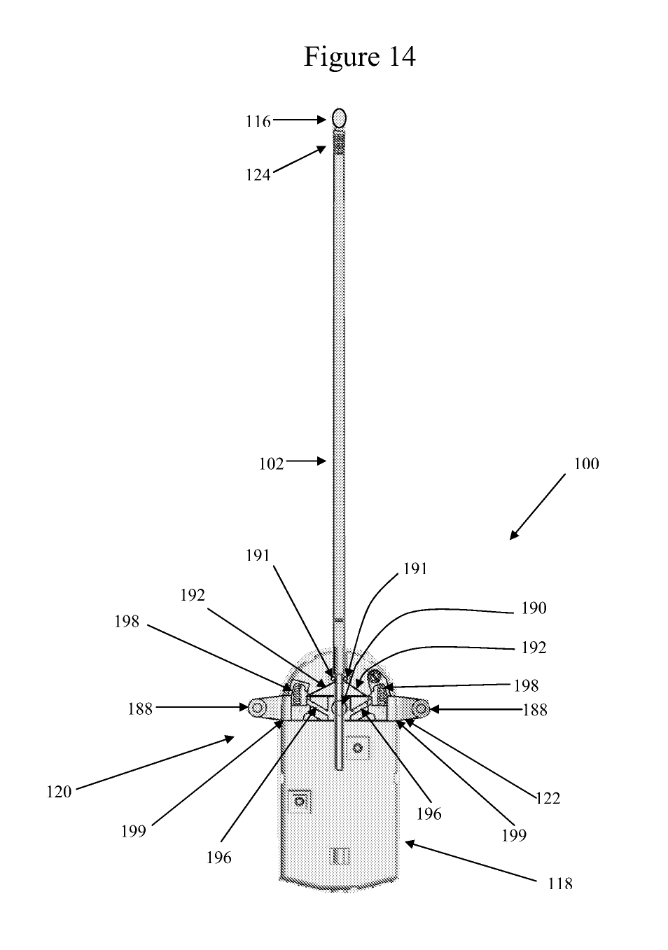

Referring still to FIG. 1, the steering mechanism 120 is configured to cause bending of the shaft 102 at one or more bending regions 124. In FIG. 14, the steering mechanism 120 is depicted with the port tubing and a portion of the housing 118 of the balloon cannula device 100 removed. The steering mechanism comprises a lever 122 that is configured to rotate or pivot at a lever axle 190. The lever 122 is attached to two control members 192 that are slidable located along the length of the shaft 102 and are attached at a distal location of the shaft 102. One or more posts 191 may be provided against the control members 192. In some embodiments, the posts 191 may be facilitate changes in the orientation of the control members 192, smooth sliding of the control members 192, and/or to protect other components of the balloon cannula device from cutting or other damage caused by the movement of the control members 192. In some embodiments, the ends of the control members 192 are secured to the lever 122 in one or more retaining channels or retaining structures, but in other embodiments, the control members may be proximally attached to form a control member loop that may be secured to a lever by placing the loop within a retaining channel of the lever. In some embodiments, one or more control members 192 or the control loop may be crimped, wound, sutured and/or embedded into the lever. The movement range and force may be augmented by one or more bias members 198 acting upon the lever 122. The bias members 198 may comprise helical springs as depicted in FIG. 14, but may also comprise leaf springs or any other type of bias member configuration. The movement range of the lever 122 may also be affected by the size and/or configuration of the lever openings 199 provided in the housing 118. In some embodiments, an optional locking mechanism may be provided to substantially maintain the lever in one or more positions.

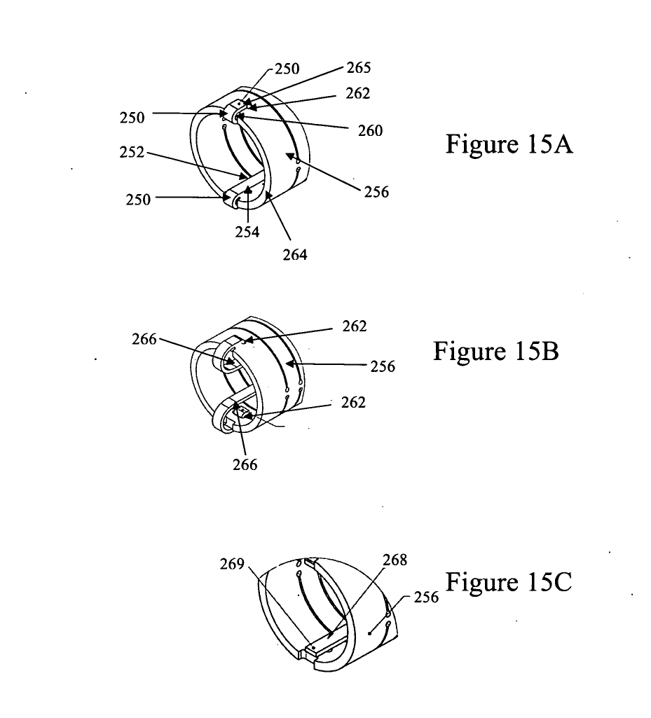

The control members 192 may comprise wires, threads, ribbons or other elongate structures. The flexibility and/or stiffness of the control member 192 may vary depending upon the particular steering mechanism. In further embodiments, the characteristics of the control member 192 may also vary along its length. In embodiments comprising two or more control members, the control members need not be configured symmetrically, e.g. having the same length, cross-sectional area or shape, or opposite attachment sites with respect to the longitudinal axis of the tubular shaft. Also, individual control members need not have the same configuration along their lengths. For example, although the proximal end of the control members 192 depicted in FIG. 14 comprises wire-like members, the distal ends 250 of the control members 252, illustrated in FIG. 15A, comprises a ribbon structures 254. In some embodiments, the greater surface area of the ribbon structures may reduce the risk of damage to the flex region 256 of the cannula device 258. In the particular embodiment depicted in FIG. 15A, the ribbon structures 254 have a U-shaped configuration that forms a mechanical and/or interference fit with the flex region 256 or other distal or flexible region of the tubular shaft. The flex region 256 may comprise one or more notches 260, recesses or openings 262 configured to accept the ribbon structure 254. In FIG. 15A, notches 260 are provided to resist slippage of the ribbon structure 254 along the lip 264 of the flex region 256, while the openings 262 are provided to permit insertion of the ribbon ends 264 to further augment the interfit of the ribbon structures 254 and the flex region 256. FIG. 15B illustrates another embodiment where in the ribbon structure 266 inserts through the opening 262. In this particular embodiment, the ribbon structure 266 may also be welded or soldered back onto itself to form a loop to further secure the ribbon structure 266 to the flex region 256. In other embodiments, as depicted in FIG. 15C, the tip 269 of the ribbon structure 268 may be bonded or soldered to the flex region 256 or the tubular shaft, depending upon the material of the ribbon structures and the flex region or the tubular shaft.

The bending range of tubular shaft may vary depending upon the particular design. The cannula device may be configured with a one-sided or a two-sided bending range with respect to the neutral position of the tubular shaft. The bending range is from about 0 degrees to about 135 degrees, while in other embodiments, the bending range is from about 0 degrees to about 90 degrees, and sometimes about 0 degrees to about 45 degrees, and still other times about 0 degrees to about 15 or about 20 degrees. The bending range of the other side, if any, may be less than, equal to, or greater than the first side. In some embodiments, increased bending angles may cause creasing or telescoping of the tubular shaft, which may obstruct one or more channels within the tubular shaft.

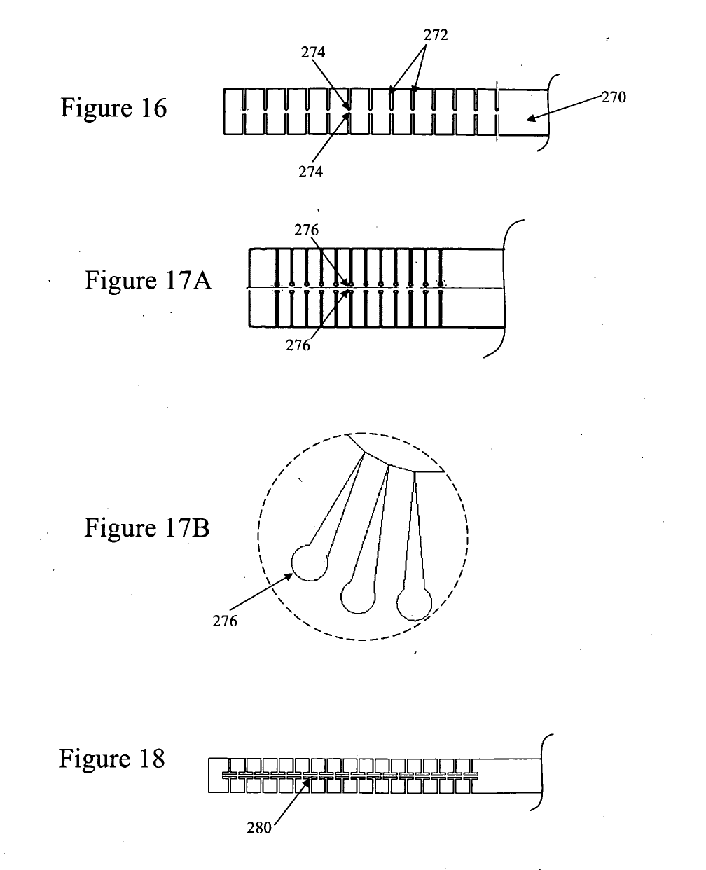

In some embodiments, to enhance the bending range of the tubular shaft, one or more flexion slots may be provided on the shaft. FIG. 16 depicts one embodiment of tubular shaft 270, comprising a plurality of slots 272. The slots 272 may have a generally circumferential orientation, but may alternatively have a helical orientation or other orientation. The slots 272 may be equally or unequally spaced along the longitudinal length of the shaft 270. In one example, the slots that are located about the ends of the flex region may be spaced farther apart than the slots located about the middle of the flex region. The slots 272 may have a similar configuration or a heterogeneous configuration. The slots 272 depicted in FIG. 15 also have a generally constant width, but in other embodiments, the width may vary along the length of the slot. The spacing between the slots ends 274 of a slot 272 may be substantially similar or different among the slots 272 comprising the flex region.

As noted in FIG. 16, the slot ends may comprise a rounded configuration, or any other configuration, including but not limited to an oval end, square end, triangular end, or any other polygonal shape for example. In some embodiments, such as the example depicted in FIG. 17A, the rounded ends 276 may have a larger transverse dimension than the width of the rest of the slot 278. In some embodiments, a rounded end may better distribute the flexion stress along the edges of the slot compared to squared or angled ends. Also, ends that are larger than the slots, such as the enlarged rounded ends 276 in FIG. 17A, may reduce the degree of compression or contact between the slot edges during flexion, which may also reduce the risk of cracking at the slot end. FIG. 17B depicts the enlarged rounded slot ends 276 of FIG. 17A in flexion. In some embodiments, the slot end may have a more complex configuration, such as the T-shaped slot end 280 as depicted in FIG. 18.

In some embodiments, the number of slots per slot region may be anywhere from about 1 slot to about 100 slots or more, sometimes about 12 slots to about 50 slots, and other times about 24 slots to about 48 slots. In some embodiments, the length of the flex region may be anywhere from about 1 inch to about 20 inches, sometimes from about 4 inches to about 10 inches, and other times about 5 inches to about 8 inches in length. In some embodiments, the outer diameter of the flex region may be about 0.05 inches to about 0.3 inches, sometimes about 0.08 inches to about 0.15 inches, and other times about 0.1 inches to about 0.12 inches. The wall thickness of the flex region may be in the range of about 0.001 inches to about 0.01 inches, sometimes about 0.002 inches to about 0.006 inches, and other times about 0.003 inches to about 0.004 inches. The slots 272 may have an average slot width in the range of about 0.004 inches to about 0.02 inches, some times in the range of about 0.005 inches to about 0.015 inches, and other times about 0.006 inches to about 0.008 inches. The spacing between the slots 272 may be in the range of about 0.015 inches to about 0.1 inches, sometimes about 0.020 inches to about 0.050 inches, and other times about 0.025 inches to about 0.04 inches. The spacing between the ends of the slots may be in the range of about 0.004 inches to about 0.05 inches, sometimes about 0.006 inches to about 0.02 inches, and other times about 0.004 inches to about 0.01 inches. The maximum transverse dimension of a slot end may be in the range of about 0.004 inches to about 0.008 inches, other times about 0.004 inches to about 0.03 inches, and other times about 0.01 inches to about 0.04 inches.

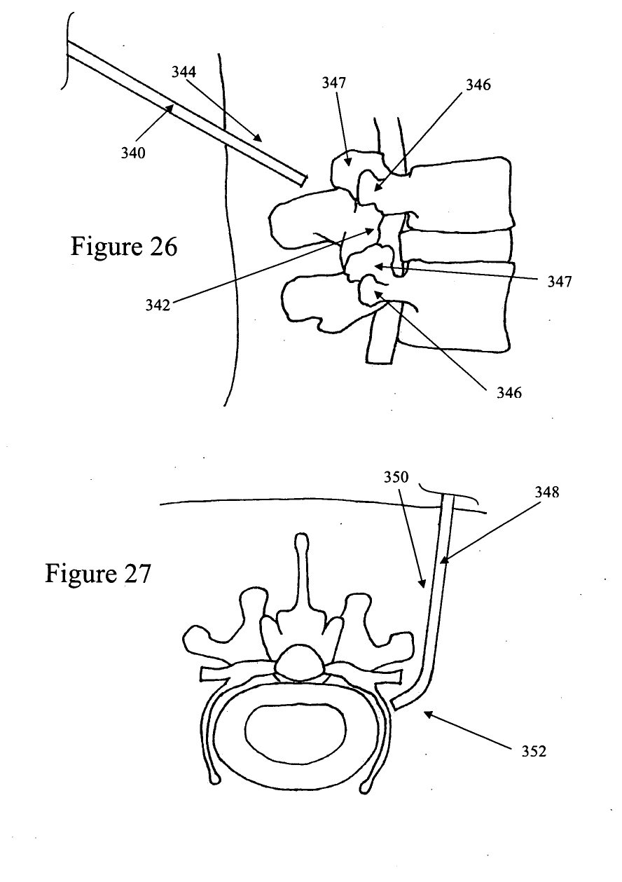

The flexion of the cannula system may facilitate access to the target site and/or reduce the degree of tissue disruption in achieving access to the target site. For example, in some procedures, the angle for approaching the target site through the skin may be different from the angle that provides the visibility or viewing angle to treat or diagnose a particular abnormality. Referring to FIG. 26, in some embodiments, a cannula system 340 may be inserted to a target site 342 by utilizing longer or indirect access pathways 344 in order to achieve the desired approach angle to a target site, and/or to avoid interference from structures such as the transverse spinal processes 346. By using a steerable cannula system 348 as depicted in FIG. 27, however, a shorter or a more direct insertion pathway 350 may be taken to a target site 352, which may reduce the aggregate degree of tissue disruption compared to a longer insertion pathway. By taking advantage of the steerability of the cannula system 348, the desired approach angle to a target site may be achieved.

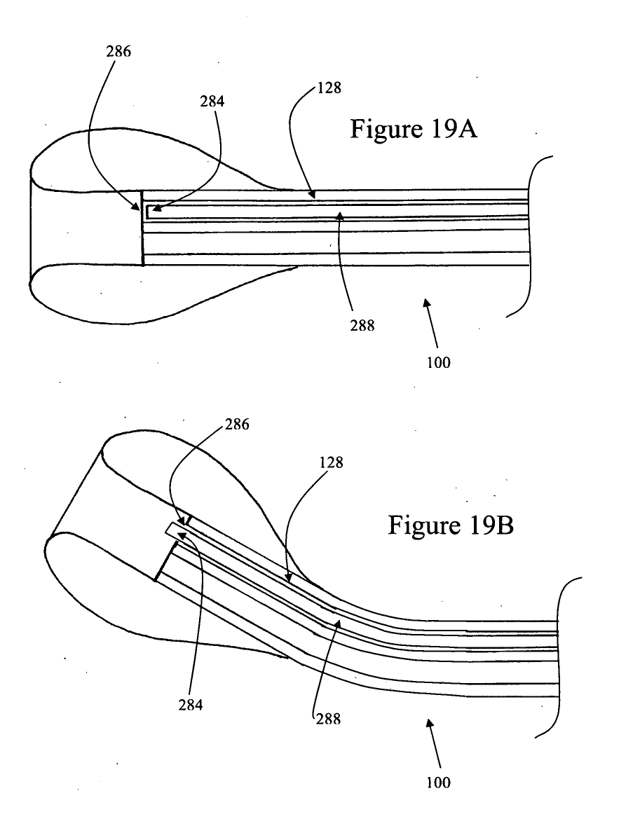

In some embodiments, during bending, one or more components inserted a channel of the balloon cannula device may exhibit different degrees of relative displacement. The degree of relative displacement may be affected by the degree of bending, the fixation or coupling site, if any between the component and the balloon cannula device, and/or the degree of displacement from the neutral position of the balloon cannula device. Referring to FIG. 19A, a balloon cannula device 100 from FIG. 5 is shown in neutral position (e.g. straight, but may be angled or curved in other embodiments) with an endoscope 282 located in the visualization port 128. The tip 284 of the endoscope 282 is in proximity to the end 286 of the visualization port 128. As the balloon cannula device 100 is flexed as shown in FIG. 19B, the tip 284 of the endoscope 282 may exhibit a relative distal displacement with respect to the end 286 of the visualization port 128, particularly in embodiments where the endoscope 282 is coupled to the balloon cannula device 100 at a proximal location (e.g. about the housing). When the balloon cannula device 100 is flexed in the opposite direction, in some instances the endoscope 282 may exhibit a proximal retraction. To compensate for the displacement, the user may manually adjust the position of the endoscope 282 as desired.

In some embodiments, the steering mechanism may also be coupled to an endoscope adjustment mechanism so that manipulation of the steering mechanism also provides at least some position adjustment which may reduce if not eliminate the degree of displacement. In other embodiments, the endoscope may be coupled to the balloon cannula device about a distal region of the tubular shaft so that, during flexion, the proximal portions of the endoscope exhibit the displacement rather than the distal portions. In still other embodiments, a spring or other type of bias member may bias the endoscope distally against an interference structure (not shown) located at the distal end of the tubular shaft to maintain the endoscope position during flexion. In some further embodiments, the interference structure may be rotated or moved out of its interfering position to permit endoscope positioning more distally, as desired.

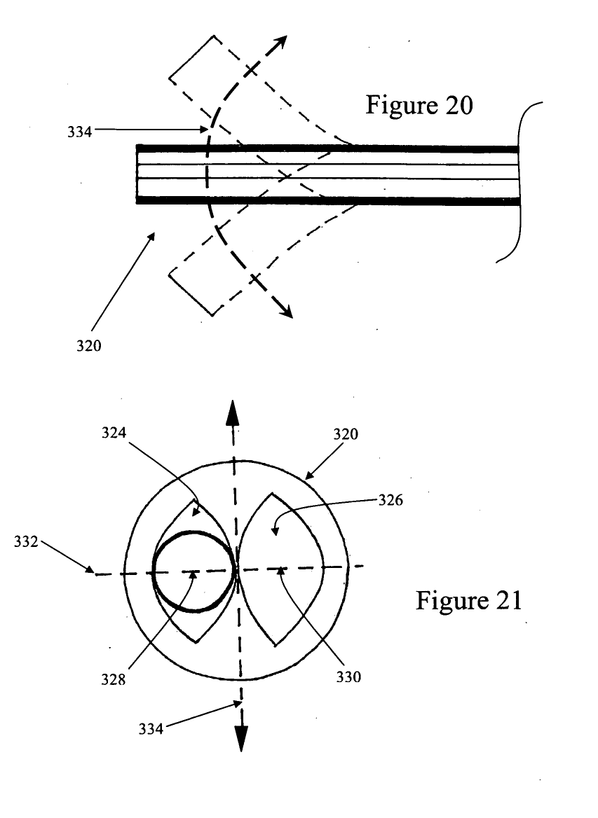

FIG. 20 is a schematic representation of a tubular shaft 320 of one embodiment of a cannula device 322 configured for two-sided flexion within a bending plane. In some embodiments, one or more channels of the tubular shaft 320 may be configured and positioned to reduce the degree of endoscope or instrument displacement during flexion. In FIG. 21, for example, the tubular shaft 320 comprises a visualization channel 324 and a working channel 326 wherein the centers 328 and 330 of the channels 324 and 326, respectively, are located along a plane 332 that is perpendicular to a bending plane 334 of the cannula device 322. Plane 332 may be located, for example, between the midpoint of the two distal attachments of the steering mechanism. The relative position of the plane 332 and the bending plane 334 may vary depending upon the particular manner in which the steering mechanism is anchored to the flexion region. In other embodiments, the centers 328 and 330 need not be located on the plane 332, but the central location of the optics or working instruments inserted into the channels 324 and 326 are located on the plane 332. For example, a channel may be configured such that the optical center of an endoscope is substantially aligned with the plane 332, even through the weighted center of the channel and/or endoscope may not be located on the plane 332 (e.g. where the lens of the endoscope is asymmetrically located, or where the central viewing angle In embodiments comprising circular channels, the center of the channel may be the center of the circle. In other embodiments comprising non-circular channels, the center of a channel may be characterized as being coaxial with the center of the largest circular object that may be inserted into the channel.

Although the embodiment shown in FIG. 21 is directed to a cannula device having a single bending plane, in other embodiments, the cannula device may be configured with two or more bending planes. With these latter embodiments, one or more channels may be aligned with one bending plane but not another bending plane. In some embodiments, a central channel may be provided that is aligned with two or more bending planes.

As mentioned previously, an endoscope or working instrument (e.g. graspers or tissue debrider) may be inserted into one or more channels of the cannula device through a proximal port. The proximal port, endoscope, and/or working instrument may be optionally configured with one or more features to lock and/or adjust the position of the inserted component. In other embodiments, one or more components of the endoscope or working instrument may be an integrally formed component of the cannula device and is not configured for removal.

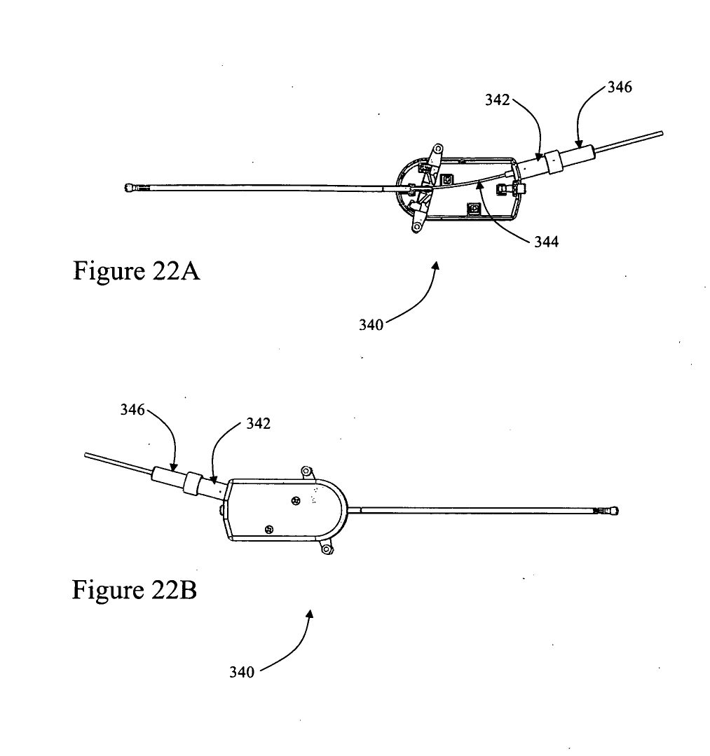

For example, in FIGS. 22A and 22B, a balloon cannula device 340 is configured with a scope port 342 in communication with the visualization channel (not shown) with a segment of tubing 344. The scope port 342 may comprise a lumen with a viscoelastic or friction surface material that is configured to slidably grip an inserted endoscope. The slidably grippable materials may include but are not limited to silicone, a urethane, including viscoelastic urethanes such as SORBOTHANE® (Kent, Ohio) and any of a variety of styrenic block copolymers such as some made by KRATON® Polymers (Houston, Tex.). The scope port 342 thus need not have any particular clamp or locking mechanism to secure the endoscope or working instrument to the scope port 243, nor any particular adjustment mechanism. In other embodiments, however, the scope port may comprise a releasable lock or clamp mechanism designed to couple to the endoscope or working instrument, with an optional adjustment assembly that may be used to modify the spacing between the lock or clamp mechanism and the housing.

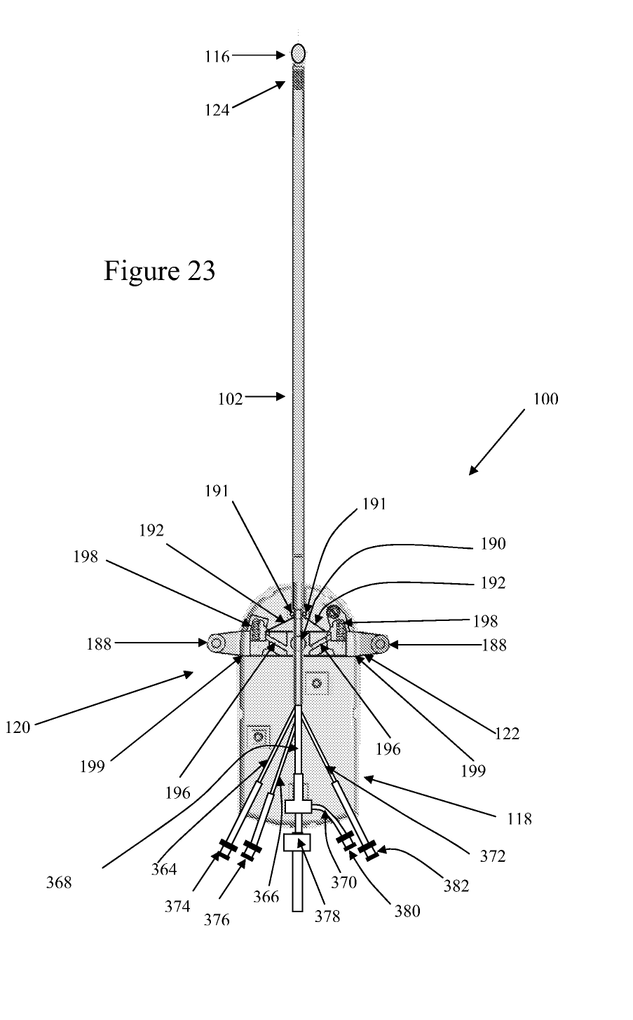

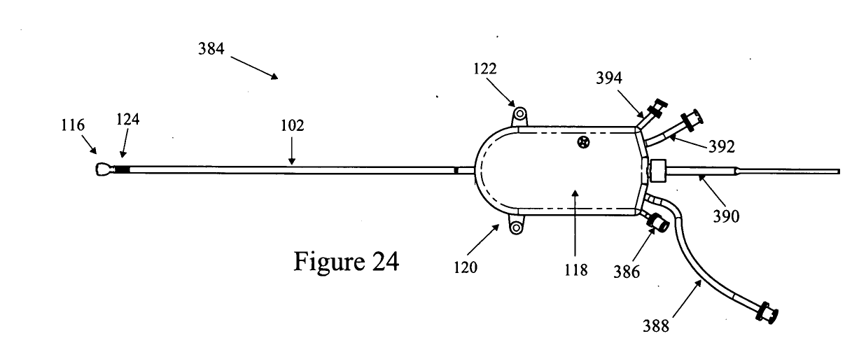

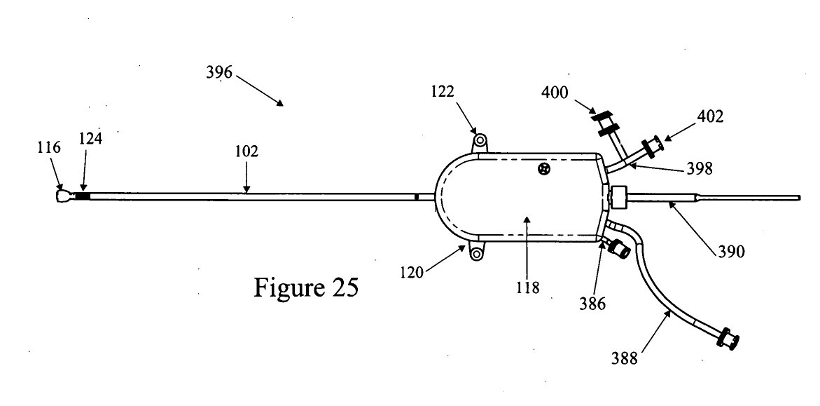

Referring now to FIG. 23, the proximal end 360 of the tubular shaft 362 may be coupled to one or more tubing segments 364, 366, 368, 370, 372 that correspond to one or more channels and connectors 374, 376, 378, and 380 of the balloon cannula device 382, respectively. As noted in FIG. 23, a tubing segment 370 may be in communication with another tubing segment, such as the tubing segment 368, which connected to the working channel of the device 382. This particular tubing segment 370 may be used, for example, to flush or aspirate fluid or material inserted into the working channel of the device 382 that is accessed through the middle port 378 and tubing segment 368. The particular design features of a tubing segment may vary, depending upon the particular function. In one example, a tubing segment used for aspiration may be rigid or reinforced to resist collapse during application of a suction source, while another tubing segment used to inflate the balloon may be configured to withstand higher positive pressures. The connector coupled to a particular tubing segment may comprise any of a variety of connectors or instrument interfaces. In some embodiments, for example, one or more connectors may comprise a standardized connector such as Luer lock, while in other embodiments, the connector may be a proprietary connectors. Depending upon the particular channel, in some embodiments, a check valve, septum, or a hemostasis valve may be provided to resist retrograde flow of fluid out of the device. The characteristics of a particular channel, including its dimensions and flexibility or rigidity, may depend upon its particular use. In FIG. 24, for example, a balloon cannula device 384 comprises five ports 386, 388, 390, 392 and 394, wherein the longer, flexible ports 388 and 392 may be used for infusion or aspiration. Such ports may be beneficial to facilitate the attachment of a bulky item such as a syringe. A rigid port, such as port 390, may be provided for instruments that may otherwise be damaged or are difficult to pass through tubing that may exhibit greater frictional resistance. FIG. 25 depicts another embodiment of a balloon cannula device 396 with a branched tube 398 with two connectors 400 and 402. This particular balloon cannula device 398 may facilitate the infusion or injection of multiple medicaments or substances that are mixed together to activate the compositions (e.g. certain adhesive-, sealing- or coating compositions).

The balloon cannula devices may be used, for example, in systems for treating disc degeneration that include nucleus decompression devices. The balloon cannula device may be used for accessing the nucleus and delivering a nucleus decompression device. For example, a decompression device may be advanced from one of the working channels of the balloon cannula device and into the nucleus of a disc. A nucleus decompression device may be used to removed the disc nucleus tissue either by dissection, suction, dissolving, or by shrinking the nucleus. Various types of thermal energy are known to shrink the nucleus such as resistive heat, radiofrequency, coherent and incoherent light, microwave, ultrasound or liquid thermal jet energies. Mechanical tissue removal devices may also be used. Decompression of the disc nucleus may result in the protruded disc material collapsing toward the center of the disc. This may reduce the pressure on the spine nerve roots, thereby minimizing or reducing the associated pain, weakness and/or numbness in the lower extremities, upper extremities, or neck region. One or more devices that may be used to strengthen and/or support the weakened disc wall may also be used with a balloon cannula device.

The combination of a balloon cannula device and a decompression device results in increased tactile sensation for the surgeon thereby allowing the surgeon to atraumatically manipulate surrounding tissue to accurately deliver the decompression device. The decompression device may be any of a wide variety of devices suited for decompressing the nucleus. By utilizing the subject balloon cannula device, nucleus decompression devices well-known in the art may be improved as a result of the real time, on-board visualization capabilities and the creation of a working area.

In additional to spinal applications, the atraumatic cannula system may also be used for a variety of other procedures. The atraumatic cannula system, including the balloon cannula systems, may be used to provide direct visualization to a variety of both bedside and surgical procedures that were previously performed blind and/or with indirect visualization. Such procedures include but are not limited to pleural biopsy, pleuracentesis, paracentesis, renal biopsy, and joint aspiration, for example. In another example, the cannula system may be used in the emergency room or trauma centers to perform peritoneal taps to diagnosis blunt abdominal trauma.

In some embodiments, the balloon cannula device may be used for diagnostic purposes. Because of the complexity of the spine, it may be more difficult to diagnose an injury than for other medical conditions. As such, the direct visualization capabilities of the subject devices may be able to accurately identify any instability or deformity in the spine. For example, the subject device may offer direct visualization of any tumors, fractures, nerve damage, or disc degeneration. In addition, the subject devices may include sensors for collecting diagnostic data, for example, sensors that measure flow, temperature, pressure, or oxygen concentration. The subject devices may also be used to remove fluid, tissue or bone samples to be used for external diagnostic tests. Additionally, the subject devices may deliver testing reagents or additional instruments for diagnosing disc degeneration and bony degeneration, for example, the subject devices may deliver electrodes for diagnosis and treatment.

In one embodiment, the balloon cannula device may be used to perform discectomy. In this particular embodiment, the patient is prepped and draped in usual sterile fashion and in a lateral decubitis or prone position. General, regional, or local anesthesia is achieved and a rigid guidewire may be inserted percutaneously to the epidural space. Guidewire placement may be performed under fluoroscopic guidance or other types of indirect visualization including ultrasound. In some instances, a small skin puncture or incision is made about 2 to 5 inches from the midline of the patient's lumbar region to facilitate guidewire insertion. A needle may also be used to facilitate guidewire passage through some tissues. The guidewire may introduced on the ipsilateral side from which the nerve impingement has been identified and at an angle of about 25 degrees to about 45 degrees to the patient's back, but in other procedures, a contralateral approach and/or a different angle may be used. After confirmation of the guidewire location, a dilator may be inserted over the guidewire to enlarge the guidewire path to the epidural space. An introducer with a releasable lock may be inserted over the dilator to maintain access so that the dilator and guidewire may be removed. An endoscope or other type of direct visualization may be inserted into the scope channel of the balloon cannula device. An irrigation fluid source is connected to the irrigation port on the balloon cannula and activated to provide continuous flushing. A passive or active aspiration port or outlet port is checked for patency. The balloon cannula is inserted into the introducer and advanced toward the epidural space. Direct visualization of the epidural space may be performed with the endoscope as the balloon cannula nears the epidural space. As the balloon cannula enters the epidural space, the balloon may be manipulated (e.g. flexed and/or rotated) to orient the user and to identify the spinal nerve and for any disc or foraminal pathology. The balloon cannula device may then be inflated with about 0.5 cc of contrast material and then advanced closer to the treatment site. Where the treatment site is abutting or impinging upon a nerve, the inflated balloon may be used to separate the treatment site and the nerve and to create a working space at the treatment site. In some embodiments, a guidewire may be reinserted into a channel of the balloon cannula and advanced past the tip of the balloon toward the treatment site. For example, the guidewire may be inserted into a bulging region of the annular wall at the site of impingement. Insertion may occur before or after balloon inflation, and before or after a nerve is separated from a bulging disc surface. A tissue disrupting instrument is then inserted in the balloon cannula device and activated to mince or disrupt the tissue at the treatment site. The disrupted material may be swept away by the continuous irrigation and flush system, or may be removed from the treatment site by an aspiration assembly on the tissue disrupting instrument. A coagulation probe, if needed, may be inserted into the balloon cannula to achieve hemostasis and/or to shrink tissue. In some embodiments, the treated disc surface may self-seal due to the small size of the tissue disrupting instrument and/or the reduced pressure in that portion of the disc following removal of disc material. In other embodiments, the treated disc may be further treated to reduce any extrusion of disc material from the treatment site. A forceps or grasper instrument may also be used with the balloon cannula device to remove any extra-discal fragments. In some instances where fragments may have migrated through a foramen of the vertebrae, the size of the balloon cannula may permit advancement of the balloon cannula into or even through the foramen. Thus, the balloon cannula may be inserted into the central spinal canal from the foramen to retrieve any migrated fragments.

In another embodiment, a balloon cannula system may be utilized for any of a variety of cardiothoracic procedures, including but not limited to bronchoscopy, pleural biopsy, pleuracentesis pericardiocentesis, and pericardial biopsy. Pericardial biopsy, for example, is indicated for the investigation of a pericardial effusion. The procedure may be performed under fluoroscopic guidance or using endoscopic instruments, but is still associated with substantial morbidity, including but not limited to risks of a pneumothorax and myocardial rupture. A minimally invasive, direct visualization alternative may improve the risk/benefit profile of the procedure. In one particular embodiment, the patient is prepped and draped in usual sterile fashion. Local anesthesia is achieved in the subxiphoid region of the patient. In other embodiments, other entry points into the thoracic cavity may be used instead. In other embodiments, regional or general anesthesia may be used instead. In some embodiments where a pericardial drainage catheter was already in place, the guidewire may be inserted into the catheter and the catheter may be removed, leaving the guidewire in place. The guidewire may be a straight guidewire or a J-tip guidewire, for example. In embodiments where an initial entry into the pericardial space is made by the guidewire, a catheter may be inserted over the guidewire and one or more pericardial fluid samples may be taken for chemistry, histology, and/or culture, for example, before continuing the procedure. One or more dilators may be inserted over the guidewire and removed to widen the tissue pathway from the skin to the pericardial space. After widening the guidewire pathway, the balloon cannula system may be inserted over the guidewire. In some embodiments, as the balloon cannula system is inserted, a sampling of the parietal pericardial tissue (i.e. the outer pericardial surface) may be taken before or after the placement of the balloon cannula system into the pericardial space. In some embodiments, the balloon may be inflated and pressed against the parietal pericardial surface. A grasper may be used to take one or more tissue biopsies of the parietal pericardial surface. A coagulation probe may be used to provide hemostasis following the biopsy or biopsies. The balloon cannula may be deflated and advanced distally over the guidewire toward the pericardial space. Once in the pericardial space, the guidewire is optionally removed from the balloon cannula system. The pericardial fluid may be drained and replaced with saline or a gas to facilitate viewing. In patients with a hemorrhagic effusion, additional irrigation and/or drainage may be used to improve the clarity of the viewing field. The balloon may be inflated and the pericardial space may be explored by flexing and/or rotating the balloon cannula device. In some embodiments, the balloon cannula may be flexed in a retrograde fashion and the inflated balloon tip of the balloon cannula is used to atraumatically tent up the pericardial tissue to reduce the tissue laxity and increase the success of the biopsy. Unlike traditional endoscopic procedures, which are sometimes contraindicated when there is insufficient fluid or loculated fluid in the pericardial sac, use of the balloon cannula system may facilitate tissue separation between the pericardium and the epicardium to safely perform the biopsy in those situations. Tissue biopsies of the visceral pericardium and/or the epicardium may be taken using graspers or other endoscopic biopsy tools. Using a tissue debrider and/or a coagulation probe, one or more windows or fenestrations may be formed in the pericardium to provide ongoing drainage of the pericardial effusion. Pericardial windows or fenestrations, if any, may be performed before or after entry into the pericardial space. The balloon cannula may then be removed and an x-ray may be taken to check for a pneumothorax. If needed, chest tube drainage may be provided until the pneumothorax has resolved.

In another embodiment, the balloon cannula system may be used to perform any of a variety of genitourinary and OB/GYN procedures, including but not limited to cystoscopy (with or without bladder biopsy), renal biopsy, prostate biopsy and surgery, fetoscopy (including optional fetal blood draws), and bladder neck suspension procedures. In one particular example, cystoscopy may be performed using a flexible balloon cannula system with a forward-positioned inflatable balloon, but in other embodiments, a rigid balloon cannula system may also be used. In one embodiment, a cystoscopy procedure may be performed by draping a patient in the usual fashion and prepping the urethral orifice with a sterilizing agent and a topical anesthetic. In patients where ureteroscopy may be performed in addition to cystoscopy, regional or general anesthesia may be used instead. A topical anesthetic is optionally applied to the exterior of the balloon cannula system as the balloon cannula system is inserted into the urethral orifice and advanced to the bladder cavity. In some embodiments, the bladder may be filled with a gas or a liquid to expand the bladder wall for viewing. Once in the bladder, the balloon cannula system may be flexed and rotated to view the bladder cavity. Biopsies may be taken as indicated by inserting a biopsy instrument (e.g. graspers) into a channel of the balloon cannula device, actuating the biopsy instrument and withdrawing the biopsy instrument. The ureteral orifice may be identified and the balloon cannula may be inserted into the ureter. A guidewire may be optionally inserted through the balloon cannula system and into the ureteral orifice to facilitate passage of the balloon cannula system into the ureter. In some embodiments, the balloon of the balloon cannula system may be at least partially expanded during entry and/or advancement of the device, to reduce the risk of ureteral perforation. Depending upon the length of the balloon cannula system, the balloon cannula system may also be advanced into the intrarenal collecting system. If a stone is encountered during the procedure, a basket or other type of capturing instrument may be inserted into the balloon cannula system to remove the stone. For stones that are too large to be withdrawn through a channel of the balloon cannula system, a burr or other type of disrupting structure may be used to break up the stone. Once the biopsies and/or stone break-up or removal is completed, the balloon cannula system may be withdrawn.

It is to be understood that this invention is not limited to particular exemplary embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a blade” includes a plurality of such blades and reference to “the energy source” includes reference to one or more sources of energy and equivalents thereof known to those skilled in the art, and so forth.

The publications discussed herein are provided solely for their disclosure. Nothing herein is to be construed as an admission that the present invention is not entitled to antedate such publication by virtue of prior invention. Further, the dates of publication provided, if any, may be different from the actual publication dates which may need to be independently confirmed.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. For all the embodiments described herein, the steps of the method need not be performed sequentially.

What is claimed as new and desired to be protected by Letters Patent of the United States is:

1. A minimally invasive spinal endoscopy system, comprising:
a tubular shaft comprising a slotted flexion zone, at least two slidable control wires, a proximal end, a distal end, at least two irrigation channels, an inflation channel, at least one non-circular instrument channel, and a visualization channel, wherein the tubular shaft has an average diameter of less than about 3.5 mm;
a movable actuator attached to at least two slidable control wires;
a housing enclosing the proximal end of the tubular shaft and at least a portion of the movable actuator; and
an inflatable balloon comprising:
an extruded tubular polymeric material comprising a folded section and post-extrusion reoriented polymer chains;
a proximal attachment to the tubular shaft and a distal attachment to the tubular shaft, wherein the spacing between the proximal attachment and the distal attachment has a fixed distance;
a proximal end that is proximal to the distal end of the shaft;
a distal end that is distal to the distal end of the tubular shaft;
an open-ended common balloon lumen between the distal end of the shaft and the distal end of the inflatable balloon, wherein the open-ended common balloon lumen has a length of at least 1 mm and is in communication with at least two irrigation channels and at least two non-circular instrument channels; and
a balloon cavity in communication with the inflation channel of the tubular shaft; wherein the inflatable balloon has a substantially cylindrical uninflated configuration and a substantially toroidal inflated configuration having a diameter that is about three to about six times greater than the longitudinal length of the open-ended common balloon lumen when the inflatable balloon is inflated to at least about 60 psi;
wherein the average diameter of the open-ended common balloon lumen decreases by less than about 15% from the uninflated configuration to the inflated configuration at a pressure of at least about 60 psi.

2. The minimally invasive spinal endoscopy system of claim 1, further comprising an endoscope comprising a shaft having an average diameter of less than about 1 mm and configured for insertion into the visualization channel.

3. The minimally invasive spinal endoscopy system of claim 2, wherein the visualization channel has a smaller cross-sectional area than at least one instrument channel.

4. The minimally invasive spinal endoscopy system of claim 1, further comprising a guidewire, a dilator, an introducer sheath, a tissue debrider, a grasper, a coagulation probe, and an infusion cannula configured for insertion into at least one instrument channel.

5. A minimally invasive device for use in a body, comprising:
a tubular body comprising a proximal end, a distal end, a first lumen therebetween, and an inflation lumen; and
an inflatable member comprising an inflation chamber in communication with the inflation lumen of the inflatable member, a proximal end, a distal end, a balloon lumen therebetween that is in communication with the first lumen of the tubular body;
wherein the proximal end of the inflatable member is proximal to the distal end of the tubular body and the distal end of the inflatable member is distal to the distal end of the tubular body; and
wherein the inflatable member has a base unexpanded configuration and an expanded configuration.
